(19) United States
(12) Patent Application Publication
(10) Pub. No.: US 2024/0387057 A1
(43) Pub. Date: Nov. 21, 2024

(54) SYSTEMS AND METHODS FOR PREDICTING CARDIAC RISK OF ANOMALOUS AORTIC ORIGIN OF CORONARY ARTERY

(71) Applicants: The Research Institute at Nationwide Children's Hospital, Columbus, OH (US); Georgia Tech Research Corporation, Atlanta, GA (US)

(72) Inventors: Rajesh Krishnamurthy, Dublin, OH (US); Lakshmi Prasad Dasi, Dublin, OH (US)

(21) Appl. No.: 18/665,768

(22) Filed: May 16, 2024

Related U.S. Application Data
(60) Provisional application No. 63/502,524, filed on May 16, 2023.

(51) Int. Cl. G16H 50/50; G16H 10/60; G16H 50/30

(52) U.S. Cl. CPC G16H 50/50; G16H 10/60; G16H 50/30

(57) ABSTRACT

A computer-implemented method for predicting risk of ischemia in anomalous aortic origin of a coronary artery (AAOCA) includes receiving medical imaging data of a patient. The method includes extracting, from the medical imaging data, patient-specific morphological imaging biomarkers pertaining to AAOCA and incorporating the biomarkers into a computer model. The method includes simulating, using the computer model, hemodynamics of the patient under simulated stress conditions and a combination of variables, the variables comprising physiological properties of the patient. The method also includes predicting and outputting a patient-specific risk profile of ischemia and/or sudden cardiac death based on simulated hemodynamics.

### CROSS-REFERENCE TO RELATED APPLICATIONS

The present application claims the benefit of priority under 35 U.S.C. § 119 from U.S. Provisional Patent Application No. 63/502,524 entitled “Predictive Model of Sudden Cardiac Death in Anomalous Aortic Origin of Coronary Artery (AAOCA),” filed on May 16, 2023, the disclosure of which is hereby incorporated by reference in its entirety for all purposes.

### TECHNICAL FIELD

The present specification generally relates to systems and methods for predicting cardiac risk of anomalous aortic origin of coronary artery (AAOCA), and more specifically relates to predictive model of sudden cardiac death in AAOCA.

### BACKGROUND

Despite being the most common detectable cause of Sudden Cardiac Death (SCD) in children and young adults, AAOCA is poorly understood. SCD is hypothesized to occur from decreased coronary blood flow resulting in myocardial ischemia and/or ventricular tachyarrhythmias and may be associated with complex and dynamic factors. No studies have assessed the specific mechanism of SCD in this population, especially in children.

Coronary angiography performed during cardiac catheterization is not helpful in the evaluation of these patients since the unique nature of the coronary ostium and proximal intramural course of the coronary artery within the wall of the aorta is not optimally characterized by this technique. Invasive studies like intravascular ultrasound and fractional flow reserve (FFR) assessments are rarely performed in children due to the higher risk associated with such procedures in the younger population. This has further limited understanding of the mechanism of ischemia.

Screening studies of middle and high school athletes reveal increasingly diagnosing coronary anomalies. Current risk stratification involves morphological and functional assessment, followed by therapeutic decisions regarding observation, exercise restriction or surgery. However, the lack of consensus or evidence on specific anatomical substrates constitutes a high-risk condition, and the lack of correlation between functional studies and outcomes resulting in lack of clarity of candidacy for each intervention. There are significant variations in specialty guidelines and controversy about risk stratification approaches.

Modeling approaches to AAOCA have not been comprehensive or useful for real-time implementation. Although biomechanical modeling has been done, it is based on a scientific foundation established in studies on atherosclerosis and valvular disease in adults, not in children. Previous biomechanical modeling approaches to AAOCA have been plagued by assumptions that have not been validated. Computed tomography (CT) based Fractional Flow Reserve (FFR) has been proposed to study AAOCA, but solutions used for fixed stenosis of coronary artery disease do not account for the biomechanical and dynamic basis for ischemia in AAOCA, and do not use patient-specific morphological data that are relevant to AAOCA like ostial anatomy and characteristics of the proximal coronary course. Recent predictive models of ischemia in AAOCA utilizing computational fluid dynamics (CFD) or fluid-structure interaction are limited in scope and are computationally intensive and expensive.

There is consensus on offering surgery for interarterial AAOCA-L, and for AAOCA-R associated with documented evidence of ischemia. Surgery is increasingly offered for the more common right coronary artery arising from the left coronary sinus (AAOCA-R) without ischemia based on “high-risk” anatomy, including long intramural course and ostial stenosis, without guidance regarding preoperative risk, whether surgery is directed at the specific mechanism that increases risk, and how surgery may alter that risk. Furthermore, surgery for AAOCA is not benign. Surgery for AAOCA in young patients may infrequently result in reoperation, decreased ejection fraction, aortic insufficiency, postoperative ischemia and death. There is a critical need for a safer and more effective solution for determining risk of ischemia, prognosis, surgical candidacy and treatment outcome in this population.

The description provided in the background section should not be assumed to be prior art merely because it is mentioned in or associated with the background section. The background section may include information that describes one or more aspects of the subject technology.

### SUMMARY

According to certain aspects of the disclosed technology, systems and methods are provided for predicting risk of ischemia and/or sudden death in anomalous aortic origin of a coronary artery (AAOCA).

According to other aspects of the present disclosure, a computer-implemented method for predicting risk of ischemia in anomalous aortic origin of a coronary artery (AAOCA) includes receiving medical imaging data of a patient. The method includes extracting, from the medical imaging data, patient-specific morphological imaging biomarkers pertaining to AAOCA and incorporating the biomarkers into a computer model. The method includes simulating, using the computer model, hemodynamics of the patient under simulated stress conditions and a combination of variables, the variables comprising physiological properties of the patient. The method also includes predicting and outputting a patient-specific risk profile of ischemia and/or sudden cardiac death based on simulated hemodynamics.

According to other aspects of the present disclosure, a non-transitory machine-readable storage medium comprising machine-readable instructions for causing a processor to execute a method for predicting risk of ischemia in AAOCA, the method includes receiving medical imaging data of a patient. The method includes extracting, from the medical imaging data, patient-specific morphological imaging biomarkers pertaining to AAOCA and incorporating the biomarkers into a computer model. The method includes simulating, using the computer model, hemodynamics of the patient under simulated stress conditions and a combination of variables, the variables comprising physiological properties of the patient. The method also includes predicting and outputting a patient-specific risk profile of ischemia and/or sudden cardiac death based on simulated hemodynamics.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 illustrates biomechanical mechanisms of ischemia/SCD in the AAOCA population.

FIG. 2 shows exemplary diagnostic criteria for mechanistic hypotheses for SCD in AAOCA refined based on pressure, flow rate, caliber, and flow patterns.

FIG. 3A and FIG. 3B illustrate an exemplary predictive model of proximal AAOCA highlighting biomechanically relevant morphological parameters and fluid structure interaction governing pressure drops at the ostia and the intramural segment in relationship to the heart and systemic circulation.

FIG. 4 shows that the equations in FIG. 3B governing the biomechanical fluid structure interaction incorporate the fundamental elements unique to the AAOCA morphology extracted from patient-specific CT including the ostial configuration, intramural course and dynamic compression related to the intercoronary pillar or aortopulmonary interaction.

FIG. 5 and FIG. 6 show exemplary simulated results based on patient data with exemplary effects of intimal wall thickness on coronary flow and pressure along the ostium and intramural segment, demonstrating a heart rate threshold where sudden drop in coronary flow and pressure occurs.

FIG. 7 and FIG. 8 show exemplary simulated results of changing intramural length while holding other parameters constant.

FIG. 9 shows exemplary simulated hemodynamics for a patient specific condition demonstrating normal instantaneous wave-free ratio (iFR) at baseline and positive stress induced iFR drop with left main coronary flow instability during simulated exercise.

FIGS. 10-15 show exemplary results of flow experiments on a patient with a short intramural course and a thickened pillar and needed recurrent surgery (e.g., coronary re-implantation) for persistent ischemia after the initial unroofing surgery (FIGS. 10-12), with comparison to a successful result in a patient with a long intramural course (FIGS. 13-15), demonstrating how different procedures target different mechanisms for ischemia in AAOCA.

FIG. 16 is a table illustrating how different procedures targeting different mechanisms for ischemia in AAOCA. By highlighting the unique mechanism of ischemia in the given patient, the predictive model may have a role in determining treatment approach for different surgical procedures.

FIG. 17 is an exemplary workflow of predicting ischemic risk of anomalous aortic origin of coronary artery that may assist risk stratification, prognosis, need for surgery, type of surgery, and surgical outcome in AAOCA.

FIG. 18 shows an exemplary computer architecture for a computer system capable of executing the software components that can execute the exemplary method/process described herein.

FIGS. 19-21 illustrate an exemplary process of creation and validation of an experimental physical 3D printed model of AAOCA that incorporates the important morphological imaging biomarkers and the material composition of the aortocoronary tissue of young patients.

FIG. 22 illustrates an exemplary aortocoronary flow model design, 3D-printed, and validated for reproduction of critical anatomical biomarkers of AAOCA using volumetric CT of the model compared to the patient's original clinical CT.

FIG. 23 illustrates an exemplary 3D-printed coupled aortocoronary and pulmonary models enclosed in a gel box to simulate the mediastinum and chest cavity in preparation for hemodynamic assessment.

FIG. 24 is a schematic of an exemplary Bi-ventricular pulse duplicator system that incorporates the 3D-printed aortocoronary model, and includes two independent flow loops with mechanically coupled pulmonary and aortic circuits.

FIG. 25 illustrates an exemplary 3D-printed patient-specific flow study results.

In one or more implementations, not all of the depicted components in each figure may be required, and one or more implementations may include additional components not shown in a figure. Variations in the arrangement and type of the components may be made without departing from the scope of the subject disclosure. Additional components, different components, or fewer components may be utilized within the scope of the subject disclosure.

### DETAILED DESCRIPTION

The detailed description set forth below is intended as a description of various implementations and is not intended to represent the only implementations in which the subject technology may be practiced. As those skilled in the art would realize, the described implementations may be modified in various different ways, all without departing from the scope of the present disclosure. Accordingly, the drawings and description are to be regarded as illustrative in nature and not restrictive.

According to certain aspects of the disclosed technology, systems and methods are provided to advance prevention science related to a significant and preventable health condition in children (e.g., 18 years old or younger) and young adults (e.g., 19-35 years old) by addressing a major research gap of understanding the mechanism of ischemia and SCD in AAOCA.

The current problem-oriented framework must be reframed to a solution-oriented approach to preventing SCD by focusing on accurate risk stratification based on the underlying mechanism of ischemia in each patient. In certain aspects, the present disclosure is the first time that a biomechanical basis for AAOCA has been explored to derive unique patient specific information that will provide accurate input for predictive flow modeling. This information is critical to developing a patient-specific predictive model of FFR based on biomarkers extracted from medical imaging data (e.g., computed tomography (CT), magnetic resonance imaging (MRI), and/or ultrasound aortic and coronary imaging data) with the potential to avoid the need for invasive FFR in children. There is broad relevance of such approaches to the field of coronary heart disease (CHD). In certain aspects, focusing on the patient-specific mechanism for ischemia improves surgical decision-making related to the need for surgery and the optimal type of surgery for a particular patient and helps to determine biomarkers of surgical success.

In certain aspects, the present disclosure provides insight into the functional outcomes after different types of surgery for AAOCA and uses a novel anatomical and mechanistic approach to explore residual risk of ischemia after surgery.

In certain aspects, the present disclosure is uniquely suited to reducing incidence of SCD in children and improving surgical decision making in AAOCA by (1) addressing the major research gap of risk stratification, (2) focusing on the patient-specific pathological mechanism for SCD, (3) creating the foundation for a predictive model of ischemia derived from medical imaging data (e.g., medical imaging data including computed tomography (CT), magnetic resonance imaging (MRI), and/or ultrasound aortic data), (4) improving decision making related to type of surgery based on underlying mechanism of ischemia, and (5) determining biomarkers of surgical success and future risk.

In certain aspects, the present disclosure includes creation and validation of patient specific 3-D printed models of AAOCA that incorporate pathological anatomy and vessel wall properties. The present disclosure is unique and paradigm-shifting in CHD in its use of biomechanical modeling using patient-specific morphology to answer questions regarding risk stratification. Pathological elements derived from standard of care imaging are incorporated to create realistic 3-D printed models with matching tissue properties that are used for hemodynamic experiments. An experimental modeling approach may benefit predictive model development in pediatric AAOCA since FFR and intravascular ultrasound (IVUS) are not part of the conventional workup for AAOCA in children. Model assessment with FFR, coronary flow, and dynamic caliber with intravascular ultrasound provides reliable data for incorporation into risk prediction models which are new in CHD and have potential for wide dissemination in adult coronary artery disease.

In certain aspects, the present disclosure bridges the evidence gap with precision-medicine approaches. In particular, the present disclosure uniquely focuses on a patient-specific biomechanical hypothesis for ischemia in AAOCA strongly supported by current evidence and will provide important insights into mechanisms of coronary ischemia and stratify the risk based on a patient's anatomy. These findings have obvious implications in the decision to treat and how to treat. The present disclosure also addresses concerns around persistent morphological (34% prevalence) and symptomatic (46% prevalence) findings after surgery and is designed to help improve surgical decision-making regarding the type of surgery and the risk factors for postoperative ischemia.

In certain aspects, the present disclosure includes unique study design to target a rare pediatric anomaly. In particular, the present disclosure uses a creative study design that exploits the synergistic nature of data-driven and mechanistic approaches that leads to a predictive model of SCD by considering all variables in space and time. Mechanistic studies can be performed on small numbers of patients. The deductive capability of mechanistic studies complements the inductive capability of data-driven approaches, allowing extrapolation to predictions about behaviors not present in the original data. The pros of one are the cons of the other, and present disclosure is directed towards a synergistic relationship.

The present disclosure develops a validated predictive FFR mathematical model that can be implemented from biomarkers extracted from medical imaging data (e.g., CT, MRI, and ultrasound data). The present disclosure delivers a medical imaging based FFR mathematical model capable of accurately assessing the risk of SCD by taking into account the various dynamic mechanisms of ischemia including sweeping through uncertainties in material properties otherwise not possible using existing methodologies of computational modeling.

In certain aspects, the systems and methods disclosed herein are configured to achieve at least five goals: (1) Targets a highly vulnerable group in terms of high-risk disease, poor understanding of patient-specific risk, difficult management decision-making and risk related to treatment, (2) Leverages the AAOCA databases on standardized imaging and surgical outcomes in a manner that does not require expensive prospective data gathering, (3) Addresses critical technological challenge of creation and validation of patient specific 3-D printed biomechanical flow models that incorporate pathological anatomy and vessel wall properties, (4) Establishes framework for a biomechanical predictive model of ischemia, greatly enhancing potential for scalability, and (5) Uses creative study design for a rare disease that exploits synergistic nature of data-driven and mechanistic approaches.

### Definitions

Unless otherwise defined, all technical and scientific terms used herein have the same meaning as commonly understood by one of ordinary skill in the art to which this invention pertains. In case of conflict, the present specification, including definitions, will control.

Unless otherwise specified, “a,” “an,” “the,” “one or more of,” and “at least one” are used interchangeably. The singular forms “a”, “an,” and “the” are inclusive of their plural forms.

The recitations of numerical ranges by endpoints include all numbers subsumed within that range (e.g., 0.1 to 5 includes 0.01, 0.05, . . . 1, 1.5, 2, 2.75, 3, 3.80, 4, 5, etc.).

The term “about,” when referring to a value or to an amount of mass, weight, time, volume, concentration, or percentage is meant to encompass variations of +1.5% from the specified amount. The terms “comprising” and “including” are intended to be equivalent and open-ended. The phrase “consisting essentially of” means that the composition or method may include additional ingredients and/or steps, but only if the additional ingredients and/or steps do not materially alter the basic and novel characteristics of the claimed composition or method. The phrase “selected from the group consisting of” is meant to include mixtures of the listed group.

Intramural coronary refers to segment of the proximal anomalous coronary artery traveling within the aortic wall, separated from the aortic lumen by the thin intimal wall. Mediastinal coronary refers to part of coronary artery after emerging from aortic wall and encased by mediastinal fat. Fractional flow rate (FFR) refers to a ratio of maximum achievable blood flow through a blockage to the flow in the hypothetical absence of blockage, calculated as pressure distal to the blockage to pressure proximal to blockage. eFFR refers to experimental FFR derived from hemodynamic assessment using 3D-printed models derived from CT. pFFR refers to predicted FFR derived from advanced imaging data using mathematical modeling. cFFR refers to clinical FFR derived from invasive catheterization of patient, at rest and after pharmacological stress. Instantaneous flow rate refers to coronary flow volume per second. The slope of its relationship with increasing pressure is a measure of coronary flow response to exercise. IVUS refers to Intravascular Ultrasound used to measure real-time, dynamic cross-sectional caliber of the coronary artery. 3D PIV or 3D PTV refers to 3-D Particulate Image (or Tracking) Velocimetry for assessing flow patterns/velocities in vitro. 4D CT/MR refers to 3-dimensional imaging with computed tomography or magnetic resonance imaging (MRI) providing multiphasic data across the cardiac cycle.

The disclosed technology includes developing an image based predictive model for exercise induced ischemia in patients with anomalous aortic origin of coronary artery. The disclosed technology uses computational modeling based on medical imaging data (e.g., CT, MRI, and ultrasound data) that results in a predictive model of ischemia. This modeling is patient specific that uses morphological risk factors. This is the first risk stratification approach for AAOCA individuals to predict who is at risk for a life-threatening event.

Herein the composite definition of ischemia may include one or more of: (1) sudden cardiac death or sudden cardiac arrest, (2) received cardiopulmonary resuscitation, (3) evidence of myocardial infarction on baseline electrocardiogram (EKG), (4) cardiac dysfunction with need for extracorporeal membrane oxygenation (ECMO), (5) ventricular tachycardia, (6) abnormal exercise stress test with ST segment depression or complex ventricular arrhythmia (e.g., ST segment on an ECG normally represents an electrically neutral area of the complex between ventricular depolarization and repolarization), (7) abnormal stress perfusion scan using sestamibi (e.g., a scan measures the amount of blood being supplied to the heart), positron emission tomography (PET) or MRI, with perfusion deficit in correct territory as anomalous coronary, (8) abnormal stress echo with wall motion abnormality in the correct territory as the anomalous coronary, and (9) abnormal cardiovascular magnetic resonance (CMR) with fibrosis or wall motion abnormality at rest or stress in the correct territory as the anomalous coronary.

### Selection Using Medical Imaging Morphological Biomarker Assessment

Without being bind by the theory, morphological imaging biomarkers like ostial stenosis, intramural course of the proximal coronary, course through a thickened pillar, and an abnormally high location of the ostium have the highest correlation with ischemia. The present disclosure shows that computed tomography angiography (CTA) reliably characterizes AAOCA when compared to surgical findings, and detects the presence and length of intramurality, ostial location, ostial stenosis and interarterial course with high accuracy and interobserver reliability. A computer-implemented process to initiate patient selection may include: (1) receiving medical imaging data, (2) performing blinded structured interpretation of CTA for high-risk morphology on all of the imaging data (e.g., CT imaging studies of the patient cohort stored in a database), and (3) generating morphological imaging biomarkers or morphological features. The morphological imaging biomarkers or features may include: coronary ostial location, ostial branching pattern, ostial shape, proximal coronary caliber, intramural length, interarterial length, thickness of the intercoronary pillar, and thickness of the intimal wall. In particular, the analysis performed for generating the morphological imaging biomarkers may include: (1) performing Principal Component analysis (PCA) to derive Z-scores of variation in imaging biomarkers in AAOCA, and (2) employing k-means clustering to characterize discriminatory power or imaging indices and proximal coronary shape for ischemia through a matching matrix.

Principal component analysis (PCA) is used to evaluate the distribution of each biomarker variation across the AAOCA cohort. PCA is an unsupervised dimensional reduction technique that condenses quantitative and shape-based features into statistical Z-scores represented as orthogonal components (modes), ranked by the amount of shape variance they explain in the AAOCA population, that quantify degrees of patient-specific shape difference from the population mean. Modes that cumulatively explain greater than 95% of the variation in the AAOCA cohort are ranked for predicting ischemic status using chi-square tests. Modes with the highest predictive score are retained for multivariate associations. Sex, age, side of anomalous coronary, coronary dominance and body surface area may be included in the models to control for differences in these confounders between ischemic and non-ischemic cohorts. Imaging indices and shape modes with the most significant associations with ischemic status after accounting for confounders are retained for clustering to discriminate ischemic status. K-means clustering, an unsupervised clustering method, is employed to characterize the discriminatory power of imaging indices and proximal coronary shape (minimum two variables required in each feature set). The ability of imaging indices and shape modes to discriminate ischemic and non-ischemic cohorts in these clusters is assessed through a matching matrix. Matthews correlation coefficient (MCC) is used to measure the quality of clinical classification.

The outcome of the morphological biomarker assessment discussed above may include: (1) prevalence weighting for identified imaging indices and shape modes that provide basis for sampling strategy for ischemic and non-ischemic groups and (2) novel imaging biomarkers of ischemia in AAOCA for risk stratification and surgical planning.

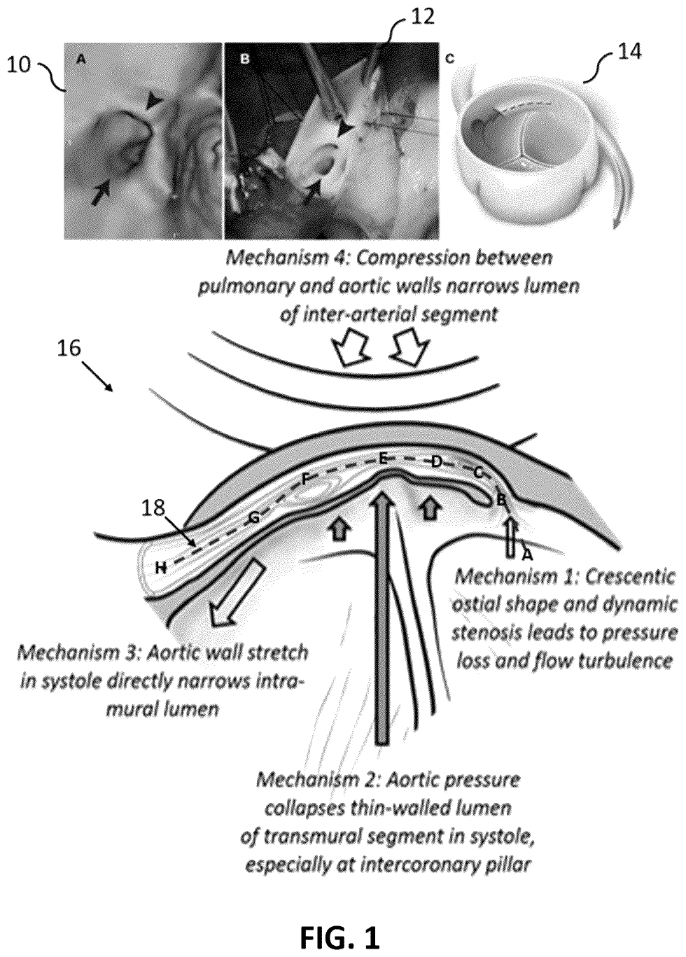

The overarching hypothesis illustrating the unique biomechanical mechanisms of ischemia/SCD in the AAOCA population is shown in FIG. 1. Images 10, 12, and 14 show the ability of CT angiography derived virtual angioscopy (image 10) to accurately reproduce the appearance of the coronary ostium at surgery in AAOCA patients (image 12). Image 14 shows the characteristic intramural course of the proximal coronary artery in AAOCA. Image 16 illustrates biomechanical mechanisms of ischemia/SCD in AAOCA, all of which are related to the unique anatomy of the ostium and proximal coronary artery and to dynamic changes to this region during exercise. CT images are only shown as a non-limiting example, biomarkers can be extracted from other medical imaging data (e.g., MRI and ultrasound data).

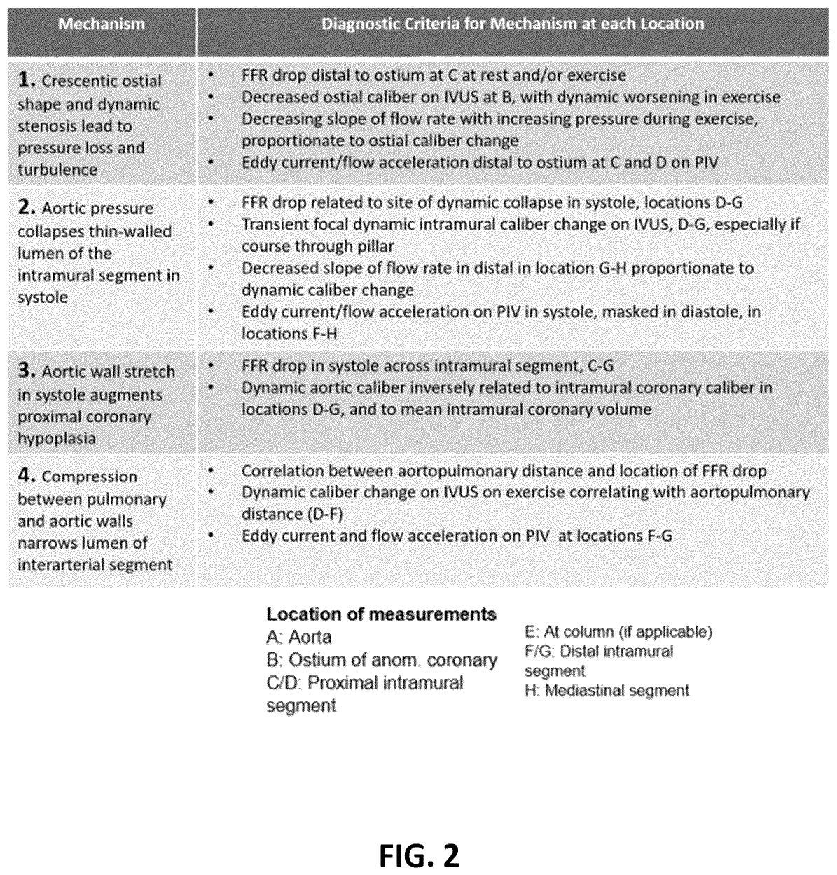

The flow field diagram in AAOCA illustrates potential mechanisms in the proximal anomalous coronary, each potentially contributing to increased resistance to coronary perfusion. The course within the wall of the aorta is termed “intramural”, while the segment that emerges from the aortic wall and is surrounded by mediastinal fat is termed “mediastinal.” Four possible underlying mechanisms of ischemia in AAOCA are illustrated in FIG. 1 and their corresponding diagnostic criteria are listed in the table shown in FIG. 2. In FIG. 2, the FFR drops are at locations A through H along a path 18 shown in FIG. 1. These mechanisms are inherently dynamic as each is related to time-varying aspects of aortic pressure, and aorto-pulmonary structural coupling within the mediastinum.

FFR has been used to assess the hemodynamic relevance of AAOCA in multiple studies. Interestingly, only a poor correlation with symptoms and/or anatomic features could be documented since the studies assessed the fixed component alone or used pharmacological stress with Adenosine stress echocardiography rather than Dobutamine echocardiography which mimics physical activity better than vasodilators. Anatomic echocardiography features like acute take-off angle or lateral compression in the intramural segment gain hemodynamic relevance during exercise, when systolic expansion and higher wall stress of the proximal aorta can be observed as a function of increasing cardiac output and systolic blood pressure. Ischemia is unlikely to occur every time the patient exercises, which suggests the presence of additional factors, such as correlation of demographic, clinical and morphological factors in AAOCA with ischemia.

With increasing age, thickening and stiffness of the aortic wall decreases distensibility, and with increasing thickness of the intimal wall of the intramural segment, the dynamic component may lose its relevance. These findings are in line with autopsy studies which reported a decreased risk for SCD beyond the age of 30. Thus, the present disclosure includes both children and young adults (e.g., restricted as <35 years to avoid the confounding effect of acquired coronary artery disease), allowing comparison of underlying biomechanics in these populations.

Predictive Model of FFR from Medical Imaging Data in AAOCA

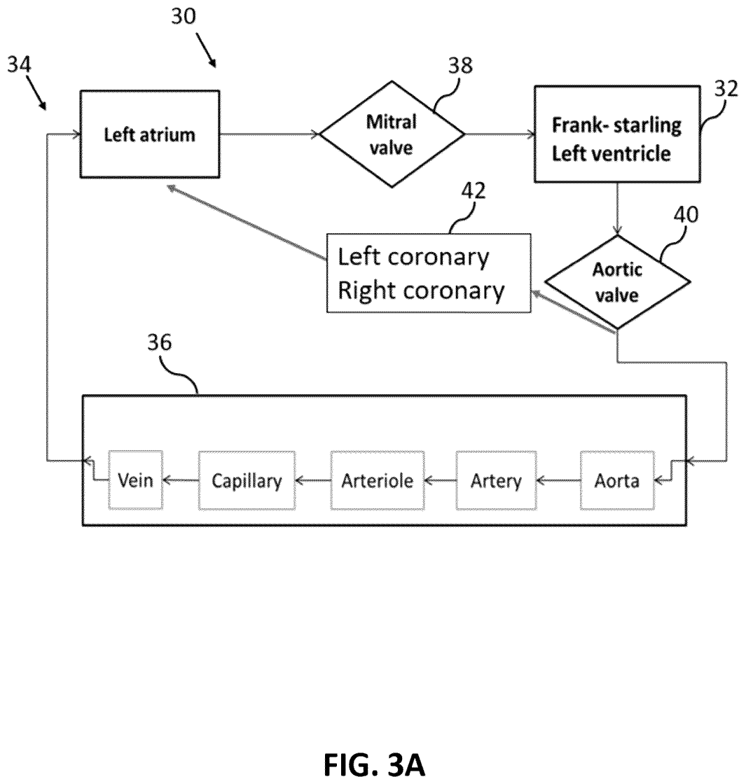

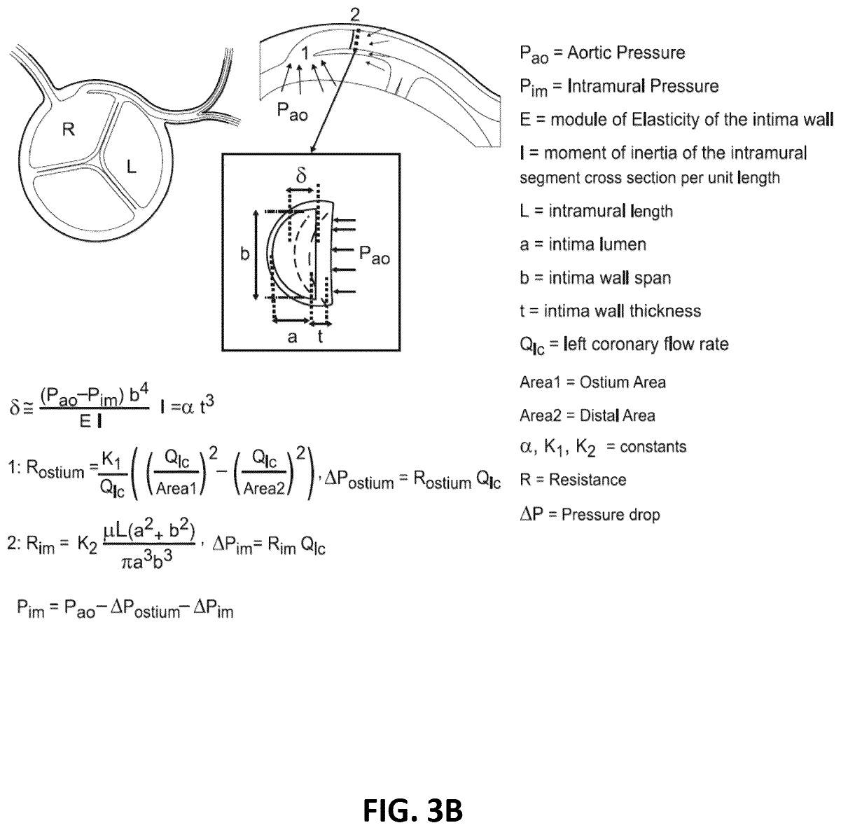

Based on the mechanistic basis discussed above, the predicted FFR derived from a mechanistic mathematical model incorporating morphological imaging biomarkers may be used to distinguish AAOCA subjects with and without ischemia. In the present disclosure, a mathematical model (e.g., a predictive model) of the left heart and systemic circulation using reduced-order lumped parameter modeling unique to AAOCA physiology is developed. The predictive model disclosed herein is configured to incorporate the medical imaging-derived AAOCA specific morphological parameters unique to each patient corresponding to the ostium, intramural length including the intimal wall thickness, and the inter-arterial segment. FIG. 3A illustrates an example predictive model (e.g., a lumped parameter model) of proximal AAOCA highlighting biomechanically relevant morphological parameters and fluid structure interaction governing pressure drops at the ostia as well as the intramural segment. The predictive model may be tuned with patient-specific clinical as well as in vitro hemodynamics.

In particular, the input is derived from patient's cuff pressure and imaging data, and includes ventricular size, aortic valve size, and coronary configuration. As shown in a systemic circulation 30 in FIG. 3A, the left ventricle 32 is driven by a time dependent elastance model following the Frank-Starling law while the systemic circulation 30 consists of a series of Windkessel elements 34 in parallel and series representing the vasculature and branching. The lumped parameter simulation of a systemic circuit 36 solves for the time variations of pressure and flow in different blocks shown in FIG. 3A which consists of the mitral valve 38 and the aortic valve 40, coronarics 42 (e.g., left and right coronaries) and the systemic circuit 36 including arteries, arterioles, capillaries, and veins. The systemic circulation 30 is modeled as a three element Windkessel block for the arterial and venous elements and the resulting aortic pressure is used to drive flow through the resistance-controlled coronary circuit with the equations governing the biomechanical fluid-structure interaction unique to the AAOCA morphology as further illustrated in FIG. 3B showing equations governing the biomechanical fluid structure interaction unique to the AAOCA morphology. The equations shown in FIG. 3B are formulated to predict the deflection of the tissue of the intramural segment wall, “δ”, for assessing the degree of underlying ischemic.

The predictive model is highly innovative at least in that it incorporates the fluid-structure biomechanics of AAOCA physiology such that the pressure drops and the corresponding incremental drops in FFR can be resolved for each morphological characteristic of the AAOCA. The predictive model incorporates dynamic deflection of the intramural segment's intimal wall driven by the pressure differential between the aortic lumen and the pressure within the intramural lumen. These dynamic deflections are then incorporated into the viscous pressure drop across the intramural segment. The predictive model disclosed herein is a highly non-linear biomechanical system that is not only calibrated on a known experimental dataset with known FFR measurements but also further tuned to recapitulate the dynamic exercise induced FFR drops at the patient-specific level. The main morphological biomarkers incorporated in the predictive model disclosed herein include: (1) ostial stenosis; (2) intramural length; and (3) dynamic narrowing due to intercoronary pillar (or interarterial compression). Each identified biomarker cluster is treated as a circuit with its own compliance, inertia and resistance. The subcircuits for the coronary circulation are also coupled to the LV pressure to respond to myocardial role in coronary hemodynamics.

### Training and Testing the Predictive Model

For each AAOCA type, the predictive model is trained by adjusting the coefficients in the model with available clinical and in vitro hemodynamic assessment. The training may be done in 3 steps:

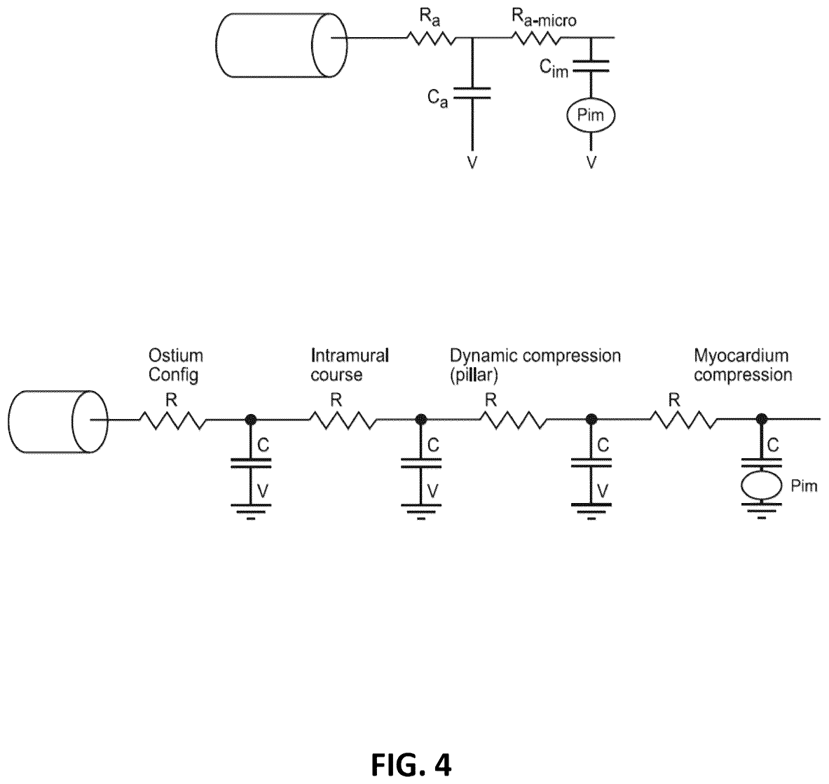

Step 1: The first set of coefficients are tuned corresponding to the constants governing the resistance parameters shown in FIG. 4 using the in-vitro training datasets corresponding to resting conditions only where high fidelity FFR is available. During this tuning process, the material property of the proximal coronary components (e.g. intimal wall) is fixed the same as the aorta.

Step 2: Once flow parameters for given material property of the proximal coronary components are tuned to yield consistent FFR with in-vitro resting conditions, the next step is to optimize the predictive power by running simulated exercise conditions over the training set for a range of variations in each morphological biomarker (+/− 20%) to account for measurement uncertainty as well as a range of variations in material property (+/− 20%) to account for natural variability from patient to patient. For each subject, the number of simulated exercise scenarios leading to ischemia (FFR<0.8) is divided by the total number of scenarios yielding the probability of exercise induced ischemia for the specific subject. Sensitivity and specificity analysis to quantify the area under the curve is performed to quantify prediction accuracy for subjects with adverse exercise-induced drop in FFR (<0.8). Once the area under the curve is obtained, the predictive model is further tuned to alter the material property variables (which are held constant in step 1) to maximize the area under receiver operating characteristic (ROC) curve. Steps are repeated for every change in material property variable until the most optimal area under ROC curve is established, and the predictive model is then deemed ready for testing.

In Steps 1 and 2, AAOCA-specific lumped parameter modeling is performed with variations in properties listed in Table 1.

TABLE 1

| | Heart | | Ostium | Intimal wall | Intramural |
|---|---|---|---|---|---|
| | rate | Systolic | area | thickness | length |
| | (bpm) | fraction | (mm²) | (mm) | (mm) |
| Default case (0) | 80 | 0.3 | 0.8 | 0.8 | 10 |
| Case 1 | 80-150 | 0.3-0.5 | 0.8 | 0.8 | 10 |
| Case 2 | 80-150 | 0.3-0.5 | 0.8 | 0.715 | 10 |
| Case 3 | 80-150 | 0.3-0.5 | 0.8 | 0.8 | 10 |
| Case 4 | 80-150 | 0.3-0.5 | 0.8 | 0.8 | 5 |

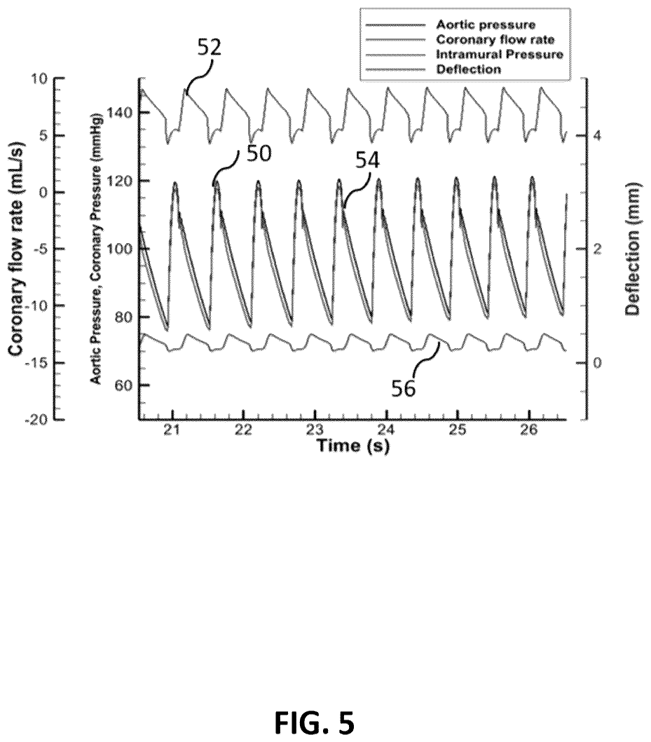

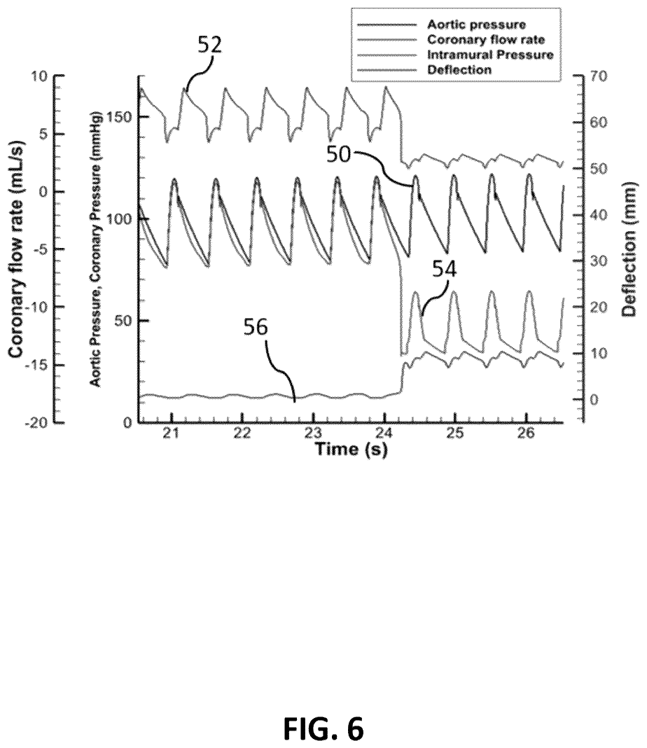

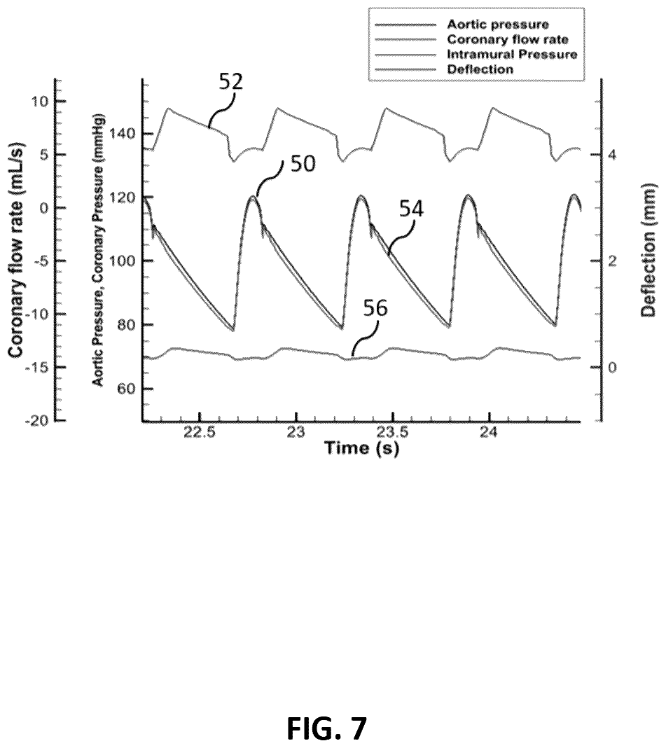

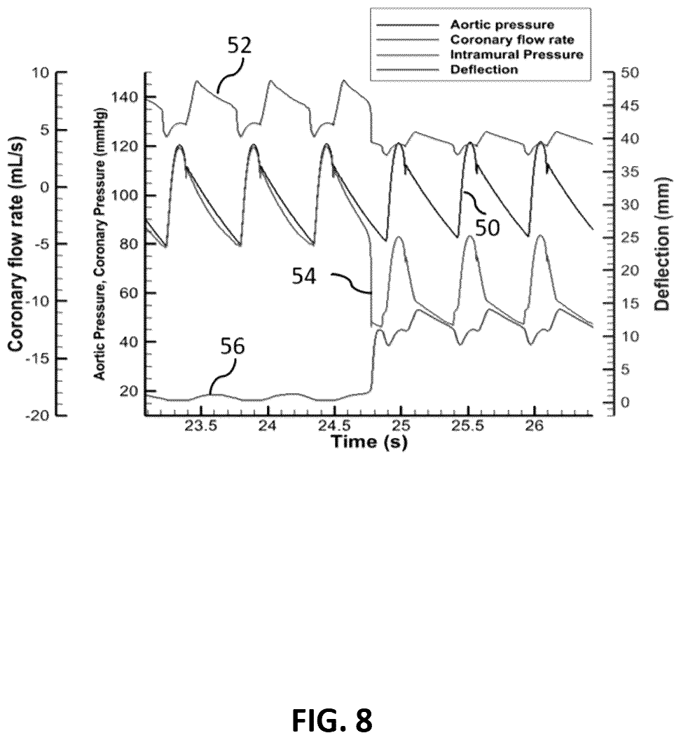

The effect of variations in morphological biomarkers such as intramural length, intimal wall thickness, and ostium shape on hemodynamics such as flow rate, pressure drop along coronary artery and resulting FFR are generated during simulated exercise. Heart rate is set at 80 bpm and gradually increased to 150 bpm (between 13^{th} second to 40^{th} seconds) for all cases and the systolic fraction is set to 0.3 (of the cardiac cycle) and increased to 0.5 at the heart rate of 120 and remained at 0.5 for higher heart rates to simulate exercise. In FIGS. 5 and 6, simulated results (using the predictive model(s) disclosed herein) show exemplary effects of intimal wall thickness on coronary flow and pressure along the ostium and intramural segment, demonstrating a heart rate threshold where sudden drop in coronary flow and pressure occurs. FIGS. 7 and 8 show simulated results of changing intramural length while holding other parameters constant.

In particular, in FIGS. 5-8, a series 50 indicates an aortic pressure profile, a series 52 indicates a coronary flow rate profile, a series 54 indicates an intramural pressure profile, and a series 56 indicates a deflection profile. In FIG. 5, there is a small difference between aortic pressure and pressure at the intramural segment. However, when decreasing the thickness in FIG. 6, the pressure drop increases with increasing heart rate, with a sudden drastic drop in flow rate and pressure occurring at a certain time (24^{th} second) which corresponds to a heart rate threshold. FIG. 7 shows slight pressure drop at a 10 mm intramural length with increasing aortic pressure, however, when the intramural length is decreased in FIG. 8, the pressure drop increases, until drastic drop in coronary flow rate occurs at a heart rate threshold. This threshold changes for different intramural lengths as it decreases at lower heart rates as intramural length is reduced.

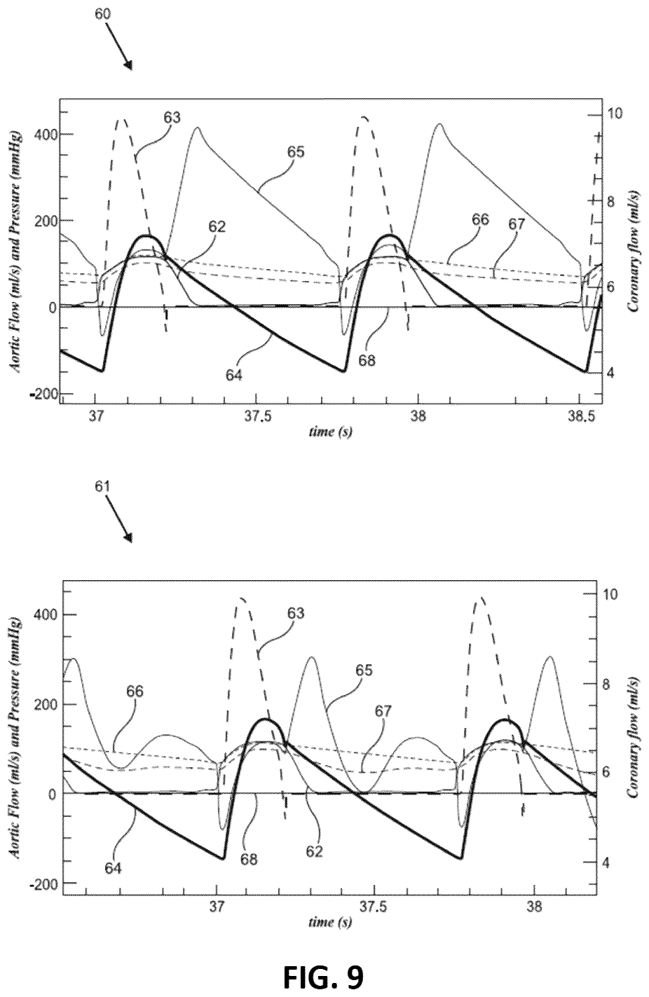

In the preliminary study in Steps 1 and 2, catheterization derived instantaneous wave-free ratio (cath-iFR) at rest and after Dobutamine stress in 8 pediatric and adult patients with AAOCA (age range 11-63 years) are collected. Morphological characteristics of the anomalous coronaries, such as ostial and proximal coronary caliber, intramural length, and intimal wall thickness are extracted from medical imaging data (e.g., CT, MRI, and ultrasound data). Baseline state (determined by the fluid parameters of the model) is tuned for each patient's corresponding resting cath-iFR. The model simulated and predicted iFR changes correspond to Dobutamine stress by tuning material properties of the intimal wall. Comparison of % predicted to measured iFR drop between rest and exercise is made in 6 patients. Resting cath-iFR range is 0.81 to 0.98 while stress iFR range is 0.72 to 0.95. Simulated iFR with stress is 0.7 to 0.94. The predictive model(s) in the present disclosure demonstrates good agreement with predicted iFR drop with stress falling within 14% error of the measured cath-iFR drop as shown in FIG. 9. In particular, FIG. 9 shows simulated hemodynamics for a patient specific condition demonstrating normal iFR at baseline shown in plot 60 and positive stress induced iFR drop shown in plot 61 with left main coronary flow instability. A series 62 indicates a left ventricle (LV) pressure profile, a series 63 indicates an aortic flow profile, a series 64 indicates a right coronary flow profile, a series 65 indicates a left coronary flow profile, a series 66 indicates an aortic pressure profile, a series 67 indicates a distal coronary pressure profile, and a series 68 indicates an intimal wall deflection profile.

Step 3: A patient-specific reduced-order approach captures dynamic changes in coronary pressure and iFR with stress. The next steps are further refinement of the model, and application in a correlation study to distinguish AAOCA subjects with and without ischemia. The hypothesis that predicted exercise-induced FFR is tested to differentiate patients with and without ischemia. The predictive model is applied to a testing dataset of N=38 patients with cFFR availability with and without exercise induced events. The hypothesis is that that there would be a probability threshold that separates those with and without ischemia. The number of patients with available cFFR is anticipated to increase over the course of the study. Comparison of pFFR with cFFR in all 100 subjects undergoing experimental studies is performed. In certain embodiments, the predictive model may be further tunned/refined to compare 50 patients undergoing eFFR evaluation before and after surgery.

Based on the studies above, correlation of pFFR with eFFR and cFFR may be derived using Bland Altman analysis. Sensitivity, specificity, predictive value of pFFR for ischemia is calculated using 0.8 as the threshold for ischemia. A computational framework in the disclosed embodiments is based on the predictive model outlined above. The computational framework is highly efficient, e.g., it takes only few seconds for each simulation, and may be further evolved to take into account sensitivity of a combination of morphological markers with respect to mechanistic effect, patient-specific myocardial and valvular interactions, physiological response, and clinical/demographic risk factors, with a potential for scalability. The computational framework disclosed herein is a non-invasive mechanistic predictive model for ischemia in AAOCA from patient's medical imaging data.

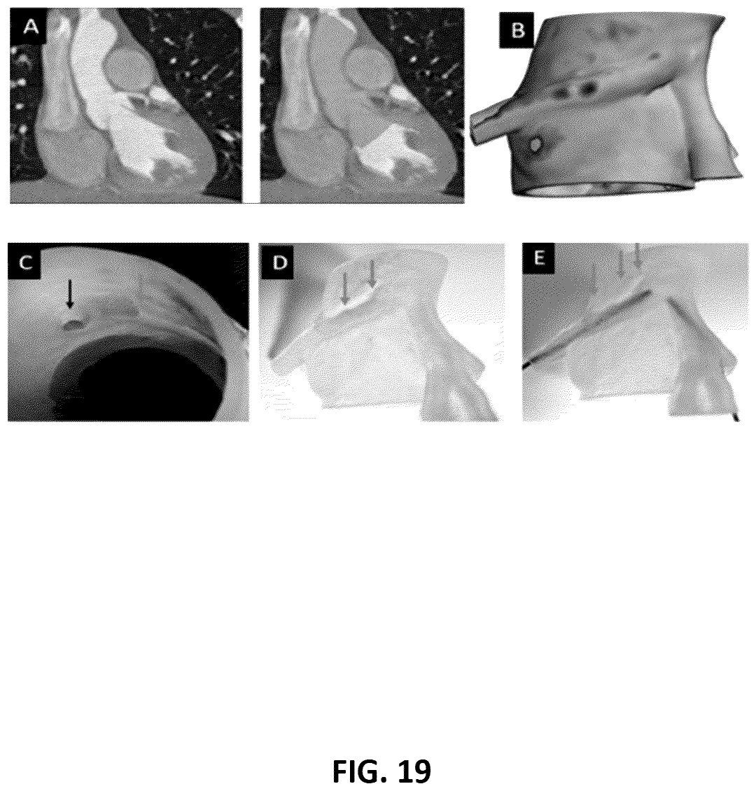

An exemplary process of developing a patient specific 3-D printed biomechanical flow models is discussed in FIGS. 19-25. The creation and validation of patient specific 3-D printed biomechanical flow models that incorporate pathological anatomy and vessel wall properties, may be utilized to validate and refine the computational framework and predictive model disclosed herein.

### Mechanistic Assessment Before and After Surgery for AAOCA

Surgical options for AAOCA include unroofing (opening the intramural segment in order to increase the size of the coronary ostium), osteoplasty (enlarging the coronary ostium), and coronary reimplantation (moving the anomalous coronary to a more normal position). The decision to operate and the type of surgery performed for the same condition can vary significantly depending on surgical approaches targeting the offending mechanism. Not every surgery is successful in relieving ischemia. The present disclosure evaluates hemodynamic effects of surgical intervention for AAOCA in the pre-operative and post-operative period and correlates result with resolution of underlying mechanism(s) for ischemia, and ultimately to predict the residual risk of SCD in these patients. Specifically, the mechanistic approach disclosed herein is capable of predicting surgical decision-making and post-operative risk stratification in AAOCA.

### Residual High Risk Anatomic Features (HRAF) on Post-Operative CTA

After unroofing, patients have no residual intramural course, but 100% have persistent acute angle of origin, 78% residual interarterial course, 49% juxtacommissural origin, and 22% have residual thickened pillar. After reimplantation, no residual HRAFs are noted, but severe proximal stenosis is present in 12% leading to surgical revision. Further resolution of ischemia was achieved in only 80% of patients, whereas 4% of patients had new-onset ischemia postoperatively. This indicates the presence of surgical risk after AAOCA repair. In addition, AAOCA patients with a short intramural segment have higher incidence of residual morphological risk factors after unroofing than patients with longer intramural segment.

Biomechanical Model Correlates with Hemodynamic Status After Surgery

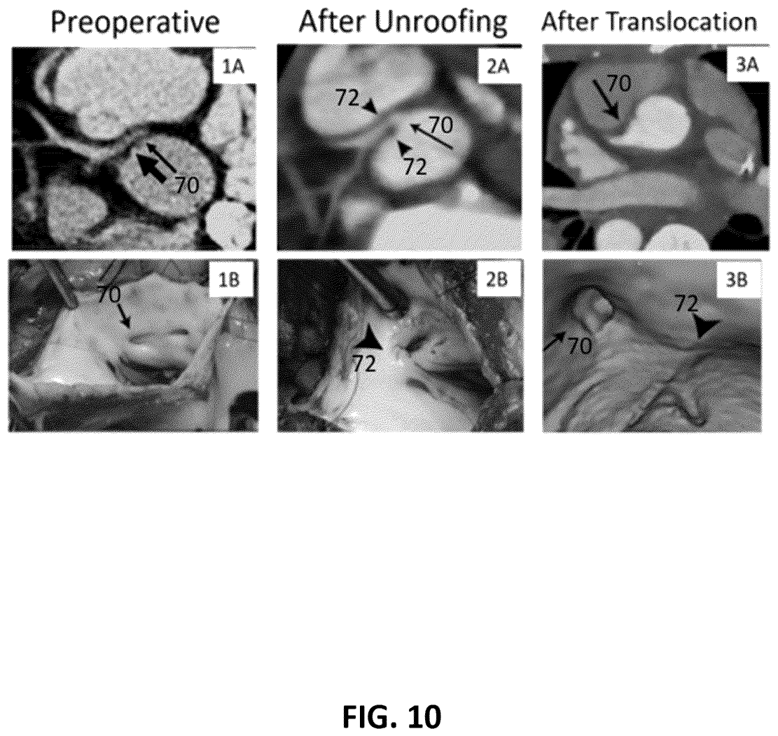

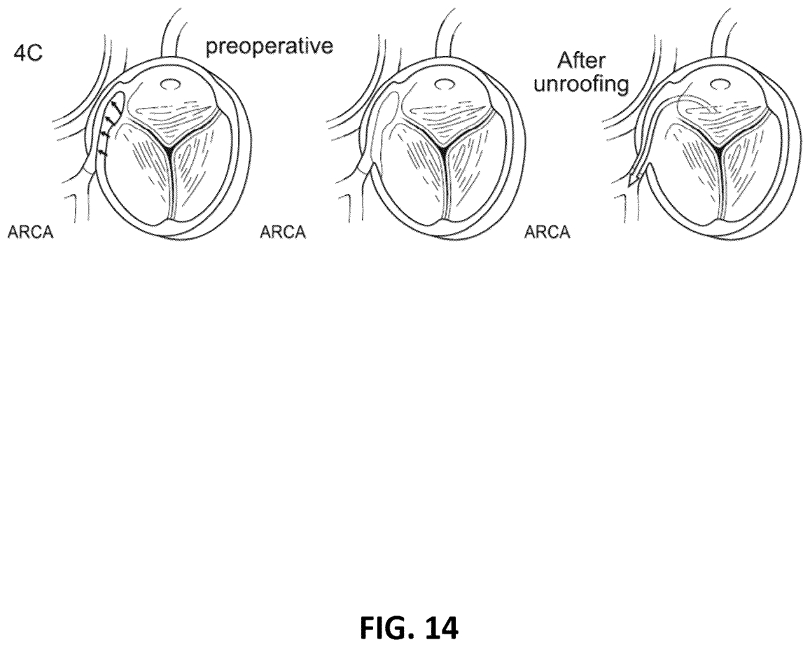

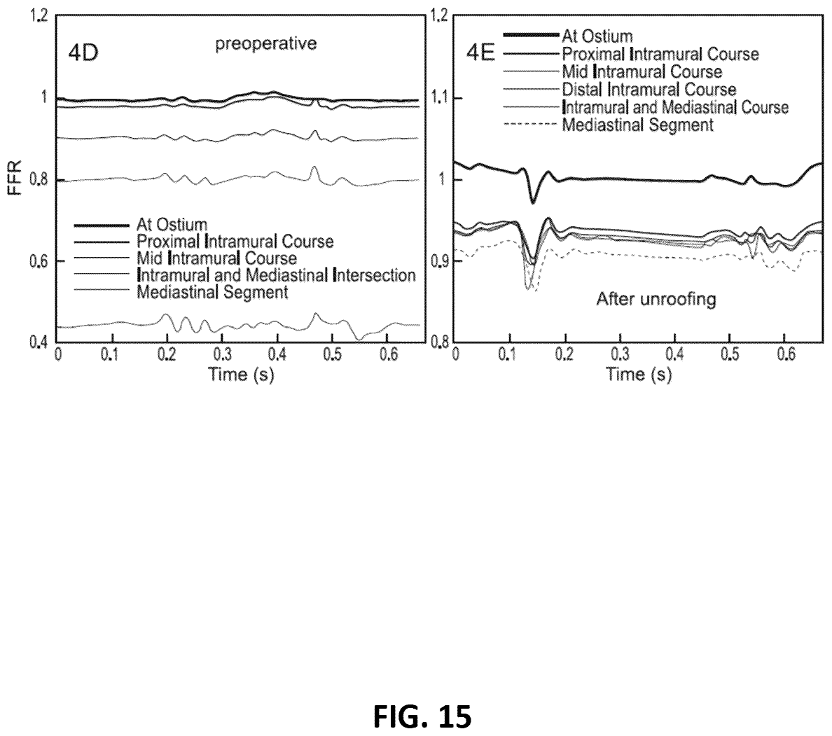

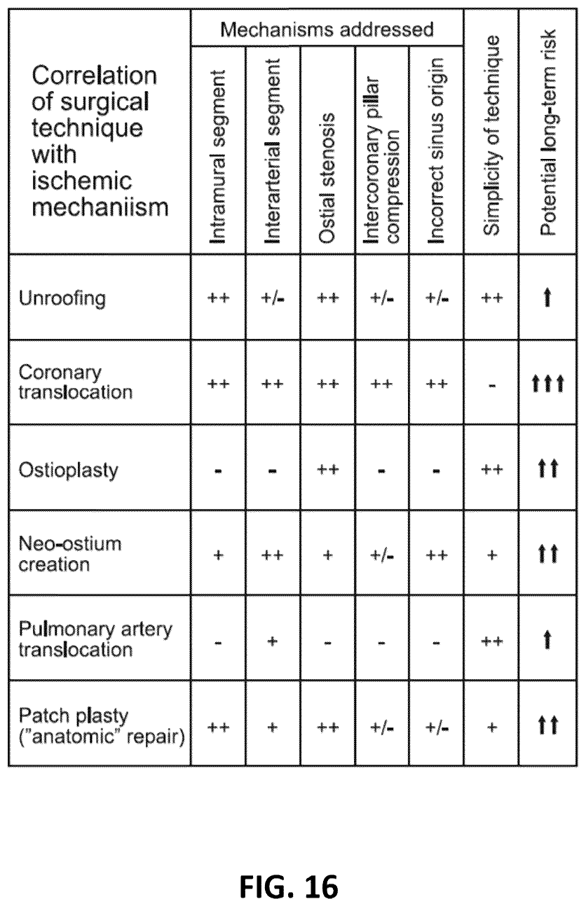

FIGS. 10-15 show results of flow experiments on a patient with a short intramural course and a thickened pillar who needed recurrent surgery for persistent ischemia, with comparison to a successful result in a patient with a long intramural course, demonstrating how different procedures target different mechanisms for ischemia in AAOCA. Through the mechanistic approach, the morphological imaging biomarkers identified and/or other risk factors post-operatively are used to predict lack of resolution of the offending mechanism for ischemia and to help guide the optimal surgical intervention to relieve those mechanisms.

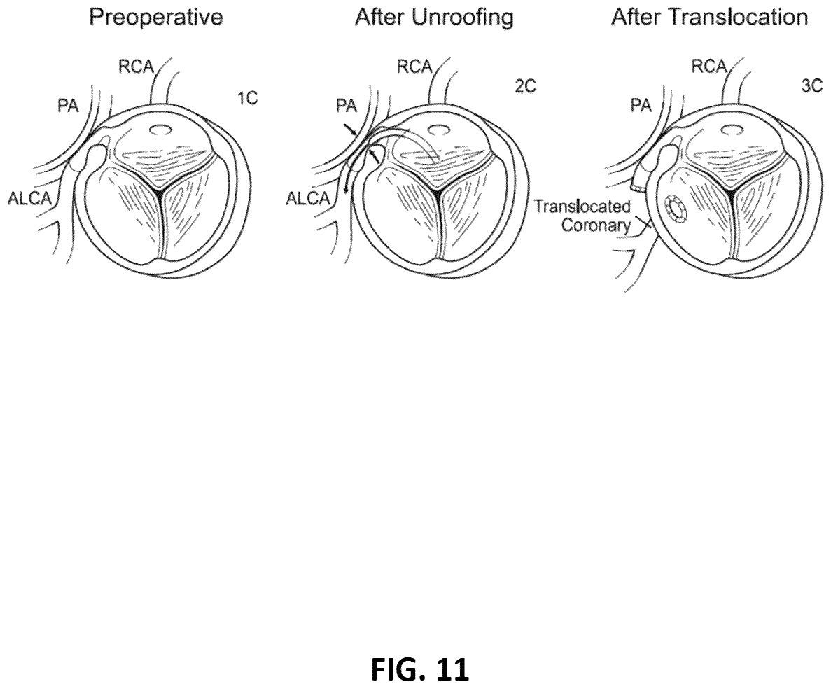

FIGS. 10-15 show the importance of resolving patient-specific mechanism of ischemia during surgery. In FIGS. 10 and 11, images 1A and 1B (preoperative), images 2A and 2B (after unroofing), and images 3A and 3B (after translocation) are from the same patient with AAOLCA and a short 3.5 mm intramural course. In FIG. 10, arrows 70 indicate ostium and arrowheads 72 indicate pillar. Initial unroofing of the short intramural segment (indicated by the arrow 70 in image 1A) increased the size of the ostium (in images 1B and 2B) and eliminated the intramural portion but not the interarterial course, predisposing to compression by the intercoronary pillar (indicated by the arrowhead 72 in images 2A and 2B).

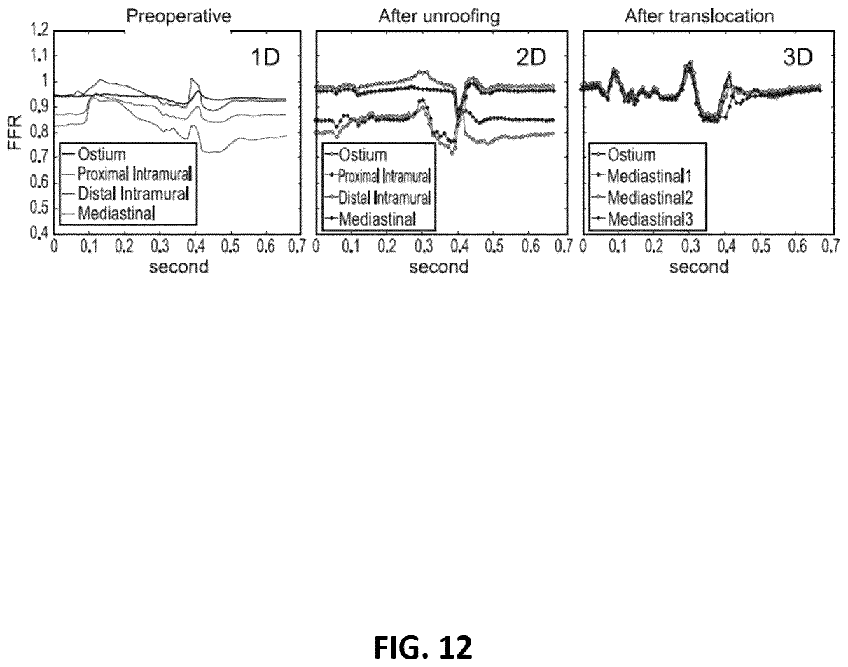

In FIGS. 10-12, images 1A, 2A, and 3A are CT images, images 1B, 2B, and 3B are intraoperative/angioscopic views of an ostium, and images 1C, 2C, and 3C are graphical representation of an anatomy. In FIG. 12, images 1D, 2D, and 3D are FFRs at different locations within proximal coronary on an in-vitro modeling study. Patient presented with repeat SCD, and a coronary translocation is performed to move the ostium to the correct sinus. Flow studies show FFR less than 0.8 in the distal intramural segment preoperatively (see image ID), FFR less than 0.8 in the mediastinal segment after unroofing (see image 2D), and normal FFR at all locations after translocation (see image 3D).

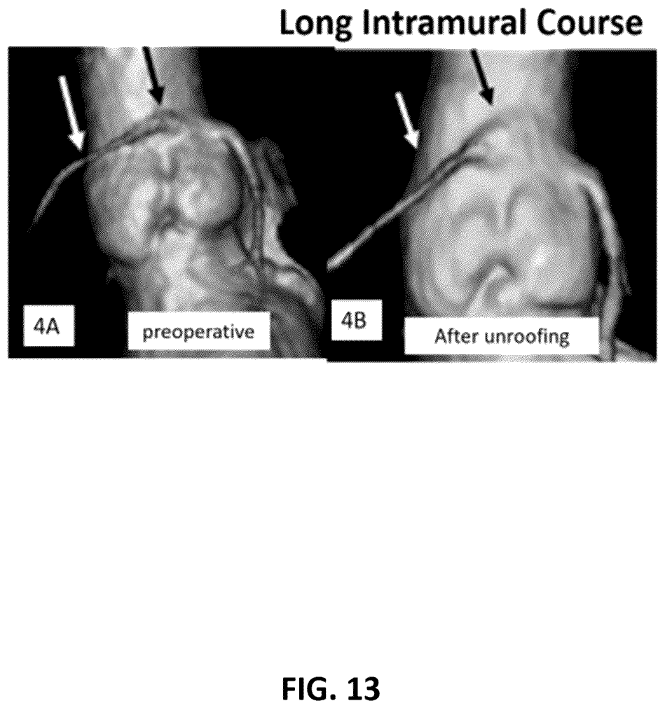

In FIGS. 13-15, images 4A, 4B, 4C, 4D, and 4E refer to a patient with anomalous aortic origin of the right coronary artery (AAORCA), a high origin, and a long 7.8 mm intramural course. In particular, images 4A and 4B refer to pre-op and post-op appearances on 3D CT after unroofing, respectively. Image 4C is a graphic of unroofing resulting in resolution of interarterial course. Before unroofing surgery, FFR falls to 0.4 on the preop modeling study (see image 4D), whereas in the post-unroofing study, FFR recovered to >0.87 (see image 4E). Based on these results, AAOCA patients with a short intramural segment have higher incidence of residual morphological risk factors after unroofing than patients with longer intramural segment.

In the present disclosure, successful results in a patient with a long intramural course, demonstrate how different procedures target different mechanisms for ischemia in AAOCA. This mechanistic study identifies morphological imaging biomarkers or other risk factors post-operatively that predict lack of resolution of the offending mechanism for ischemia and helps guiding the optimal surgical intervention to relieve those mechanisms.

Steps 1-3 for developing the predictive model described above may be carried out in the preoperative and postoperative cohort. Power calculation is based on the primary outcome. A sample size of 50 surgical patients achieves 80% power to detect an odds ratio of 1.6 and 2.2 with respect to the intra-cluster correlation of 0.7 (moderate) and 0.2 (weak correlation). The proportion of experimental ischemia after surgery is assumed to be 0.2 under the alternative hypothesis. The test statistic is effect regression coefficient from a mixed-effects logistic regression model. The significance level is 0.05.

In some aspects of the present disclosure, the outcomes before and after surgery may be compared via a generalized linear mixed model. For experimental ischemia, the exposures are: (1) the stage (at rest or with exercise) experimental ischemia is observed; (2) pre-or post-operative period; (3) the interaction term between the stage and the period; (4) pre-operative imaging biomarkers and their interaction terms with the stage and the period; and (5) type of operation and its interaction terms with the stage and the period. For coronary flow rate, the exposures are: (1) pressure; (2) pre-or post-operative period and the interaction term with pressure; (3) pre-operative imaging biomarkers and the interaction terms with pressure and the period; and (4) type of operation and its interaction terms with pressure and the period. These results have important implications regarding type of surgery, risk-benefit of surgery for asymptomatic AAOCA-R, and decision to return to full activity or exercise restriction after surgery.

In the present disclosure, in order to determine ischemia, experimental ischemia (based on 3D-printed models derived from medical imaging) may be used as the reference for mechanistic assessment due to its high fidelity and ability to recreate morphological changes related to exercise like graduated aortic pressure and distention, as well as preload and afterload effects. The results are correlated with clinical ischemic testing and cFFR where available to derive insights. The absence of ischemia on functional testing of children with AAOCA does not entirely preclude the occurrence of SCD in the future. Clinical FFR availability is sparse even in large cohorts, since FFR testing in children is considered risky and redundant in patients with clinically documented ischemia, and usually restricted to patients with confounding variables. Mode of cFFR assessment (systolic vs diastolic, resting vs adenosine vs dobutamine) may not always recreate environment for dynamic compromise, resulting in an unreliable standard.

One of the potential limitations of any biomechanical models is the reproduction of exercise physiology, specifically, the accuracy of simulating exercise in a model. This limitation applies to any testing that simulates exercise, including clinical functional studies such as pharmacological stress, including dobutamine stress MRI. To mitigate this limitation, the models disclosed in the present disclosure implement changes in the model consistent with measured physiological changes obtained from in vivo studies including preload and afterload effects. An alternative approach to biomechanical modeling may be to measure flow/pressure/IVUS caliber with catheterization in all patients, which requires prospective collection of data (impractical) and may place patients at undue risk for collection of mechanistic research data.

By focusing on the mechanism of ischemia that is patient-specific, the results obtained from the study may directly influence risk stratification of AAOCA in young patients, and in decision-making regarding need for surgery, the type of surgery, and assessment of surgical success. The computational model/framework for AAOCA disclosed herein may incorporate the biomechanical insights disclosed above and validated predictive models, as well as clinical trials to provide precise guidance which can be scaled to all clinical centers. The predictive model disclosed herein is configured to be a useful tool for rapid clinical hemodynamic assessment of patient specific risk of SCD in AAOCA and to provide patient-specific input for surgical decision making.

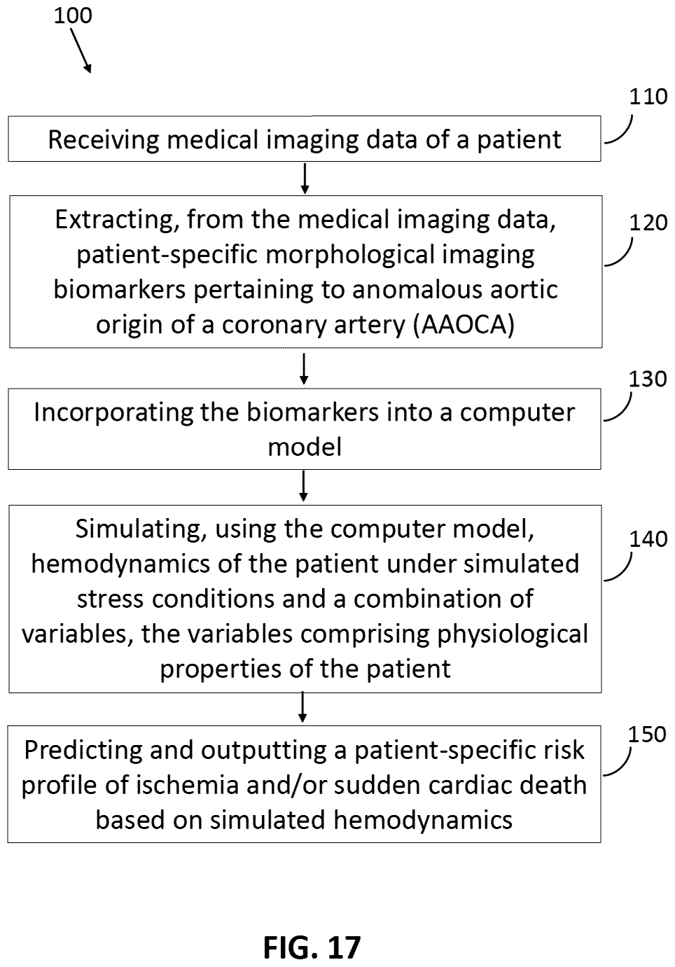

FIG. 17 shows an example computer-implemented method 100 for predicting risk of ischemia and sudden cardiac death in AAOCA. The method 100 may include receiving medical imaging data of a patient (step 110) and extracting, from the medical imaging data, patient-specific morphological imaging biomarkers pertaining to AAOCA (step 120). The method 100 may include incorporating the biomarkers into a computer model (step 130). The method 100 may include simulating, using the computer model, hemodynamics of the patient under simulated stress conditions and a combination of variables, the variables comprising physiological properties of the patient (step 140). The method 100 may include predicting and outputting a patient-specific risk profile of ischemia and/or sudden cardiac death based on simulated hemodynamics (step 150).

The computer model in step 130 may include a reduced order model that incorporates the biomarkers and solves for the hemodynamics comprising dynamic pressure and/or flow changes from ostium through distal coronary artery under the simulated stress conditions. In one example, the computer model may include one or more models discussed in FIGS. 3A and 3B. In one example, the computer model be a patient-specific Frank-Starling driven reduced order model that incorporates the biomarkers and solves for dynamic pressure changes from ostium through distal coronary artery. The Frank-Starling driven reduced order model may include a lumped parameter model of proximal AAOCA including biomechanically relevant morphological parameters and fluid structure interaction governing pressure drops at ostia and intramural segments. In one example, the computer model may include a mathematical model that performs simulation for a range of physiological vessel wall properties specific to age/sex and solves for dynamic pressure and flow changes from ostium through distal coronary artery during simulated stress. In particular, the computer model may include the mechanistic mathematical model shown in FIG. 3B formulated to predict the deflection of the tissue of the intramural segment wall, “δ” for assessing the degree of underlying ischemic.

The medical imaging data in step 110 may include computed tomography (CT), magnetic resonance imaging (MRI), and/or ultrasound aortic and coronary imaging data of the patient. Step 110 may include performing principal component analysis (PCA) on the medical imaging data to extract the biomarkers. Step 110 may include deriving Z-scores of variation in the biomarkers in AAOCA (e.g., a statistical measure that quantifies the distance between a data point and the mean of a dataset). The step 110 may include using K-means clustering to characterize discriminatory power of imaging indices and proximal coronary shape for ischemia through a matching matrix (e.g., a method of vector quantization, originally from signal processing, that aims to partition n observations into k clusters in which each observation belongs to the cluster with the nearest mean (cluster centers or cluster centroid), serving as a prototype of the cluster).

The biomarkers in step 120 may include coronary ostial location, ostial branching pattern, ostial shape, proximal coronary caliber, intramural length, interarterial length, thickness of the intercoronary pillar, thickness of the intimal wall, or a combination thereof.

The variables in step 140 may include material properties (e.g., mechanical properties) of the patient's heart and/or biomarkers (e.g., coronary ostial location, ostial branching pattern, ostial shape, proximal coronary caliber, intramural length, interarterial length, thickness of the intercoronary pillar, and thickness of the intimal wall). As the patient (e.g., a child or a young adult), these variables are expected to change. Therefore, it is important that the computer model takes takes accounts the changes in the variables (e.g., physiological properties, material properties, biomarkers) over time or over an age span.

The simulated hemodynamics in step 140 may include dynamic pressure and/or flow changes from ostium through distal coronary artery in response to the imposed stress conditions. Exemplary simulated hemodynamics, such as the the dynamic pressure and/or flow changes, are discussed in FIGS. 5-9. In some aspects, the hemodynamics may be characterized in terms of FFR, iFR, percent flow, etc. The simulated stress conditions in step 140 may include flow and/or stress conditions of the patient at rest and during exercise.

The method 100 may further include performing sensitivity analysis to determine sensitivity scores of the variables on the hemodynamics.

The method 100 may further include predicting a dominating mechanism of ischemia and/or sudden cardiac death in AAOCA based on the simulated hemodynamics. For example, as discussed in FIGS. 5-8, a sudden drop in coronary flow and pressure in the simulated results may indicate a risk of ischemia and/or sudden cardiac death in AAOCA. Mechanisms may be simulated/compared to determine the dominating mechanism(s).

The method 100 may further include outputting simulated changes in FFR, iFR, and/or percent flow for a range of physiological properties.

The method 100 may further include tunning/refining the computer-model to improve accuracy of the prediction. Machine learning including artificial intelligence (A.I.) modules or algorithms and/or machine learning (M.L.) modules or algorithms may be used to refine or train the model. As the database grows, the accuracy of the predictive model is expected to increase. 10. In some aspects, the method 100 may further include tunning the reduced order model such that a simulated iFR drop is within 14% error of a measured cath-iFR drop.

The method 100 may further include validating and refining the computer-model to improve accuracy of the prediction based on comparing simulated results with that obtained based on hemodynamic assessment using 3D-printed models derived from medical imaging data.

It should be appreciated that the logical operations, process, or method described above can be implemented (1) as a sequence of computer implemented acts or program modules running on a computing system and/or (2) as interconnected machine logic circuits or circuit modules within the computing system. The implementation is a matter of choice dependent on the performance and other requirements of the computing system. Accordingly, the logical operations described, process, or method described herein are referred to variously as state operations, acts, or modules. These operations, acts and/or modules can be implemented in software, in firmware, in special purpose digital logic, in hardware, and any combination thereof. It should also be appreciated that more or fewer operations can be performed than shown in the figures and described herein. These operations can also be performed in a different order than those described herein.

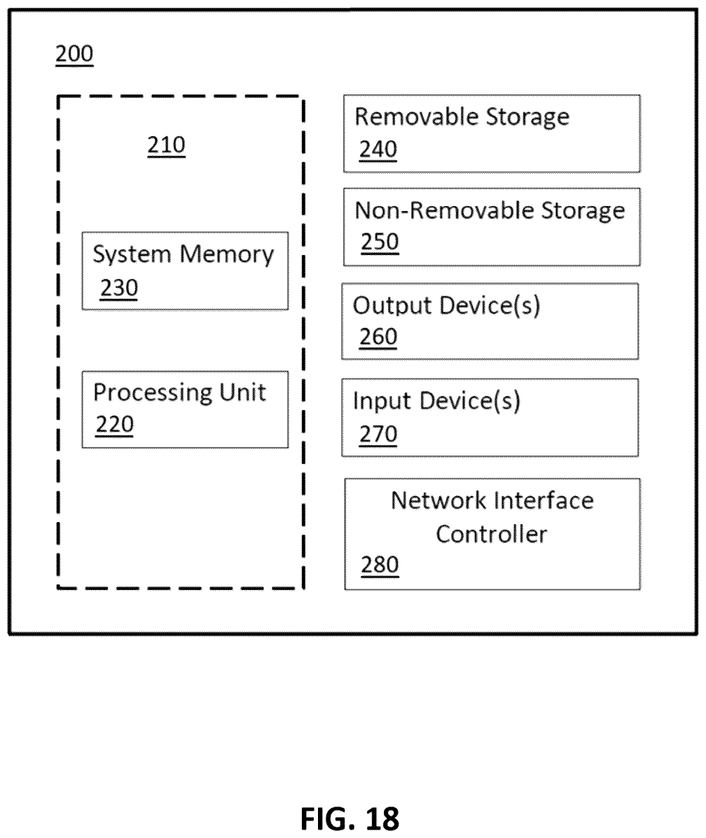

FIG. 18 shows an illustrative computer architecture for a computer system 200 capable of executing the software components that can execute the exemplary method/process described herein. The computer architecture shown in FIG. 18 illustrates an example computer system configuration, and the computer 200 can be utilized to execute any aspects of the components and/or modules presented herein described as executing on the analysis system or any components in communication therewith.

In an aspect, the computer system 200 may include two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an aspect, virtualization software may be employed by the computer system 200 to provide the functionality of a number of servers that is not directly bound to the number of computers in the computer system 200. For example, virtualization software may provide twenty virtual servers on four physical computers. In an aspect, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or may be hired on an as-needed basis from a third-party provider. Some cloud computing environments may comprise cloud computing resources owned and operated by the enterprise as well as cloud computing resources hired and/or leased from a third-party provider.

In its most basic configuration, computer system 200 typically includes at least one processing unit 220 and system memory 230. Depending on the exact configuration and type of computing device, system memory 230 may be volatile (such as random-access memory (RAM)), non-volatile (such as read-only memory (ROM), flash memory, etc.), or some combination of the two.

This most basic configuration 210 is illustrated in FIG. 18 by the dashed line. The processing unit 220 may be a standard programmable processor that performs arithmetic and logic operations necessary for operation of the computer system 200. While only one processing unit 220 is shown, multiple processors may be present. As used herein, processing unit and processor refers to a physical hardware device that executes encoded instructions for performing functions on inputs and creating outputs, including, for example, but not limited to, microprocessors (MCUs), microcontrollers, graphical processing units (GPUs), and application specific circuits (ASICs). Thus, while instructions may be discussed as executed by a processor, the instructions may be executed simultaneously, serially, or otherwise executed by one or multiple processors. The computer system 200 may also include a bus or other communication mechanism for communicating information among various components of the computer system 200.

The computer system 200 may have additional features/functionality. For example, computer system 200 may include additional storage such as removable storage 240 and non-removable storage 250 including, but not limited to, magnetic or optical disks or tapes. The computer system 200 may also contain network connection(s) 280 that allow the device to communicate with other devices such as over the communication pathways described herein. The network connection(s) 280 may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards such as code division multiple access (CDMA), global system for mobile communications (GSM), long-term evolution (LTE), worldwide interoperability for microwave access (WiMAX), and/or other air interface protocol radio transceiver cards, and other well-known network devices. The computer system 200 may also have input device(s) 270 such as keyboards, keypads, switches, dials, mice, track balls, touch screens, voice recognizers, card readers, paper tape readers, or other well-known input devices. Output device(s) 260 such as printers, video monitors, liquid crystal displays (LCDs), touch screen displays, displays, speakers, etc. may also be included. The additional devices may be connected to the bus in order to facilitate communication of data among the components of the computer system 200. All these devices are well known in the art and need not be discussed at length here.

The processing unit 220 may be configured to execute program code encoded in tangible, computer-readable media. Tangible, computer-readable media refers to any media that is capable of providing data that causes the computer system 200 to operate in a particular fashion. Various computer-readable media may be utilized to provide instructions to the processing unit 220 for execution. Example tangible, computer-readable media may include, but is not limited to, volatile media, non-volatile media, removable media and non-removable media implemented in any method or technology for storage of information such as computer readable instructions, data structures, program modules or other data. System memory 230, removable storage 240, and non-removable storage 250 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In light of the above, it should be appreciated that many types of physical transformations take place in the computer system 200 in order to store and execute the software components presented herein. It also should be appreciated that the computer system 200 may include other types of computing devices, including hand-held computers, embedded computer systems, personal digital assistants, and other types of computing devices known to those skilled in the art. It is also contemplated that the computer system 200 may not include all of the components shown in FIG. 18, may include other components that are not explicitly shown in FIG. 18, or may utilize an architecture different than that shown in FIG. 18.

In an example implementation, the processing unit 220 may execute program code stored in the system memory 230. For example, the bus may carry data to the system memory 230, from which the processing unit 220 receives and executes instructions. The data received by the system memory 230 may optionally be stored on the removable storage 240 or the non-removable storage 250 before or after execution by the processing unit 220.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language and it may be combined with hardware implementations.

Moreover, the various components may be in communication via wireless and/or hardwire or other desirable and available communication means, systems and hardware. Moreover, various components and modules may be substituted with other modules or components that provide similar functions.

The computer system 200 includes software and/or hardware components and modules needed to enable the function of the modeling, simulation, and methods disclosed in the present disclosure. In some aspects, the computer system 200 may include artificial intelligence (A.I.) modules or algorithms and/or machine learning (M.L.) modules or algorithms (e.g., stored in the system memory 230, removable storage 240, non-removable storage 250, and/or a cloud database). The A.I. and/or M.L. modules/algorithms may improve the predictive power of the models, simulations, and/or methods disclosed in the present disclosure. For example, by using a deep learning, A.I., and/or M.L. model training including patient information and any relevant input data to the computational model, the predictive power of the computational model may be greatly enhanced. The A.I. and/or M.I. modules/algorithms also help improving sensitivity and specificity of the prediction as the database grows. In some aspects, the computer system 200 may include virtual reality (VR), augmented reality (AR) and/or mixed reality display(s), headset(s), glass(es), or any other suitable display device(s) as a part of the output device(s) 260 and/or the input device(s) 270. In some aspects, the display device(s) may be interactive to allow an user to select from options including with or without AR, with or without VR, or fused with real time clinical imaging to help clinician interact and make decisions.

### Creation of an Experimental Model of AAOCA

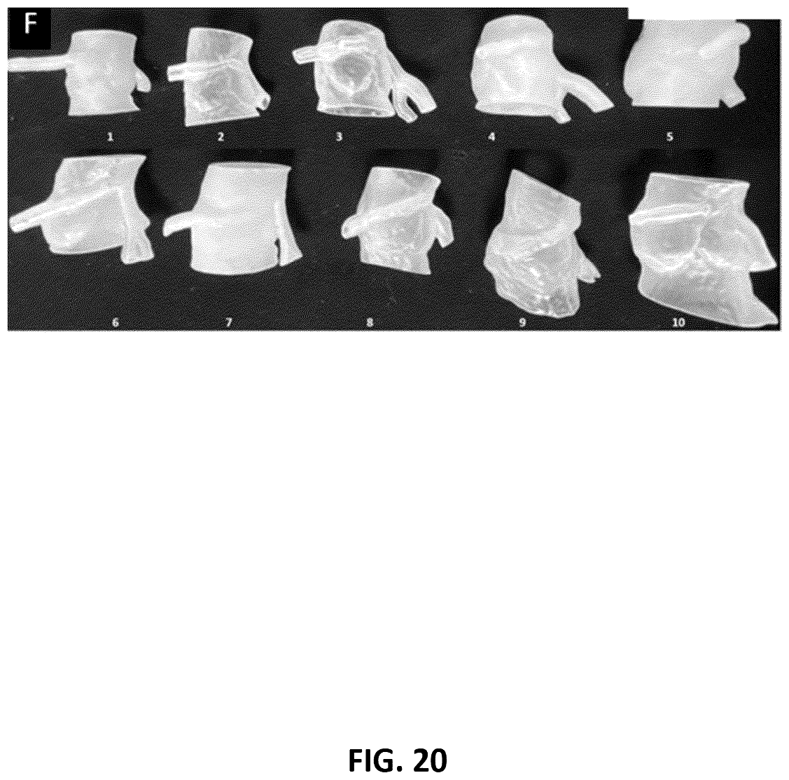

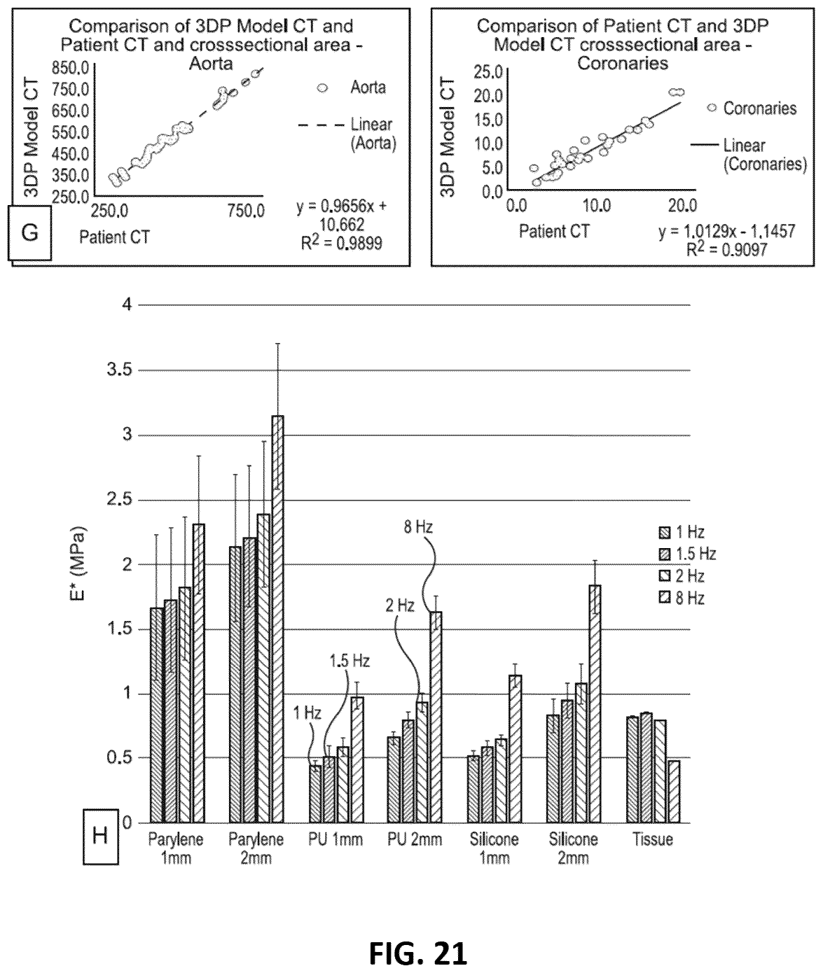

Referencing back to the exemplary process of developing a patient specific 3-D printed biomechanical flow model, the process of creation of an experimental model of AAOCA is illustrated in FIGS. 19-21. In particular, FIG. 19 compares the process of segmentation of the relevant proximal coronary anatomy from CT and creation of a computerized 3D model in the top row to the physical 3D printed model in the bottom row showing the slit-like ostium (indicated by an arrow) and the intramural course (shown as a wire). FIG. 20 shows 3D printed models from 10 different patients with AAOCA showing the ability of this approach to accurately reproduce the unique anatomy in each patient. FIG. 21 (top) shows validation results comparing the patient CT to the model CT for important morphological markers of AAOCA. FIG. 21 (bottom) shows material testing of different 3D printed materials to reproduce the biomechanical properties of aortocoronary tissue in young patients.

Due to the poor understanding of the biomechanical behavior of the proximal anomalous coronary at rest and exercise, a model that replicates patient specific anatomy and reproduces the material properties of aortocoronary wall tissue is the ideal choice to study dynamic hemodynamic behavior in this condition. Benefiting from recent advances in rapid prototyping and replication of tissue properties using polyjet three-dimensional (3D) printing, a patient-specific biomechanical 3D model is printed incorporating morphological features derived from CTA to quantify coronary blood flow in AAOCA. In particular, FIGS. 19-21 illustrate a process flow of 3D virtual model creation and verification. In FIG. 19, Image A illustrates digital imaging and communications in medicine (DICOM) CTA segmentation of aorta and coronaries and Image B illustrates a 3D virtual model of AAOCA with contours overlaid on patient CTA 3D model to screen for geometric accuracy. Image C illustrates a 3D printed AAOCA model visualization of ostia and Image D and Image E show intramural course indicated by the arrows. In FIG. 20, Image F shows 10 patient models that are studied according to the technique disclosed herein. In FIG. 21, plots in Image G show a comparison of cross-sectional area measurements between 3D printed (3DP) model and CTA for validation. Image H shows a plot of dynamic modulus values for different material composition options compared to fresh pediatric human aorta (tissue).

### Material Composition to Match Tissue Properties of Aorta and Coronary

Next, material composition is developed to match tissue properties of aorta and coronary. To determine material composition and properties, dog bone samples are designed per ASTM standards, printed with Agilus30™ (available from Stratasys) and coated with parylene, polyurethane (PU) or silicone to create desirable optical and mechanical properties, and compared to fresh pediatric human aorta specimen stored in alcohol on an Instron® machine (Instron headquartered in Norwood, MA, U.S.A.) to derive stress/strain curves and dynamic modulus and stiffness values. These models are configured to closely mimic properties of native aorta and coronary artery with dynamic modulus of about 0.5 MPa to about 1 MPa, tensile strength ranging between about 2.4 MPa and about 3.1 MPa, elastic modulus of about 0.72 MPa to about 1.47 MPa for aorta, and elastic modulus of about 0.9 MPa to about 1.6 MPa for coronary arteries. To replicate the above mechanical properties and compliance, a wall thickness (WT) of about 2 mm is assigned for aorta and about 1.5 mm is assigned for coronaries. The intimal wall of the intramural segment is directly measured from the CT image, and its exact wall thickness is assigned and validated.

### Biomechanical Model Validation

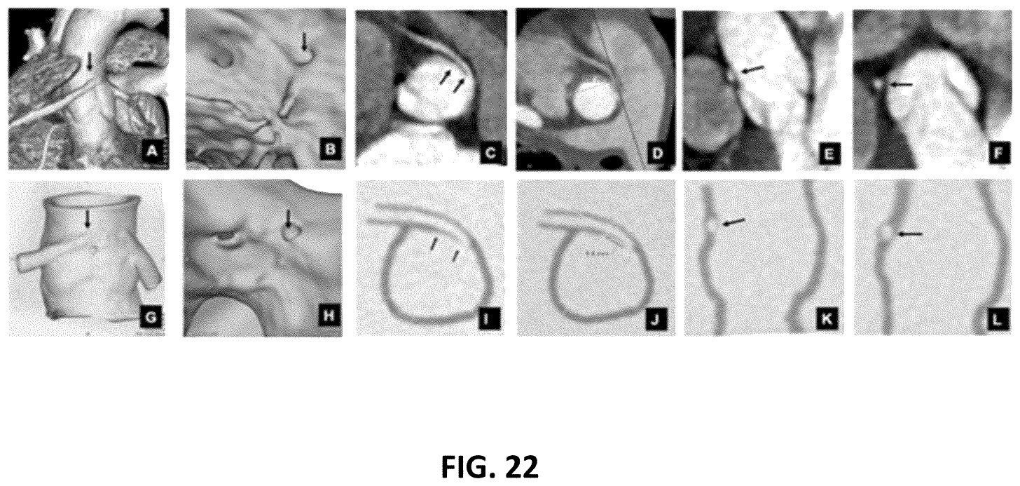

Next, biomechanical model validation is performed. Aortocoronary flow models are designed and 3D printed with Agilus 30™ and VisiJet® S500 (available from 3D Systems, Rock Hill, SC, U.S.A.), and validated using volumetric CT as shown in FIG.22. The top and bottom rows show a comparison of CTA of the coronary arteries in a 14-year-old male patient (top row) to CT of 3D printed model of the coronaries (bottom row) for reverse validation of patient-specific model for morphologic substrates in AAOCA. The dynamic cross section measurements of proximal coronaries in the intramural and mediastinal segments are identical to CTA. Pulsatile behavior of aortic and coronary walls and the intramural segment are demonstrated on 4D CT/IVUS. Images A and G are volume rendered images of the proximal coronaries. Images B and H are virtual angioscopy of the coronary ostia with slit-like ostium of RCA. Images C and I are proximal course of anomalous RCA intramural segment. Images D and J are blinded measurement of intramural length of RCA is 5.6 mm in both. Images E and K are cross section (CS) of intramural segment showing oval shape with a thin (soft tissue density in E) wall on the aortic side (indicated by an arrow). Images F and L are CS of mediastinal segment of RCA shows round shape with thicker (complete cuff of fatty density in image F) wall on the aortic side (indicated by an arrow).

Biomechanical Model Correlates with Ischemic Status in AAOCA

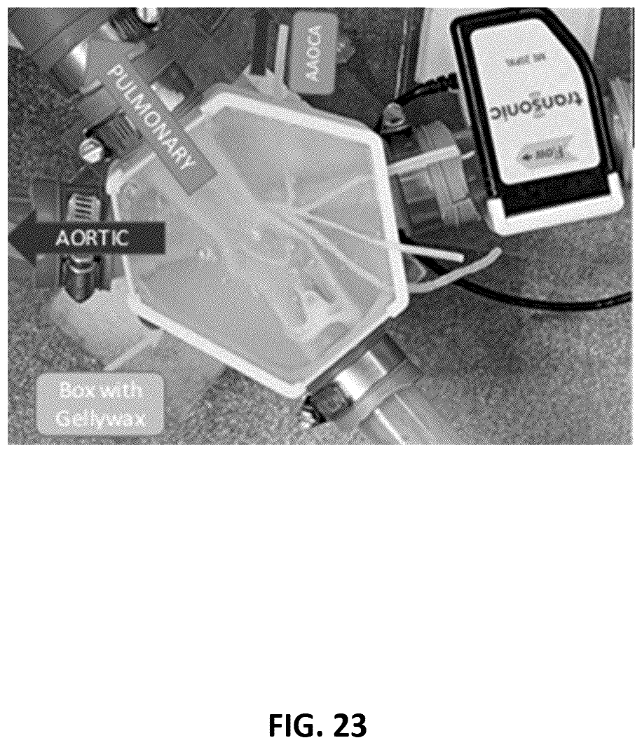

Aortocoronary models are printed with connections to inflow and outflow pipes. The process is repeated for the pulmonary artery. The model is placed in a 3D printed box and encompassed in Gellywax (available from RBC industries, Warwick, RI, U.S.A.) such that their anatomical relationship is preserved as shown in FIG. 23. The 3D printed coupled aortocoronary and pulmonary models are shown in the box enclosed in a gel box. The models are shown connected to a pulse duplicator.

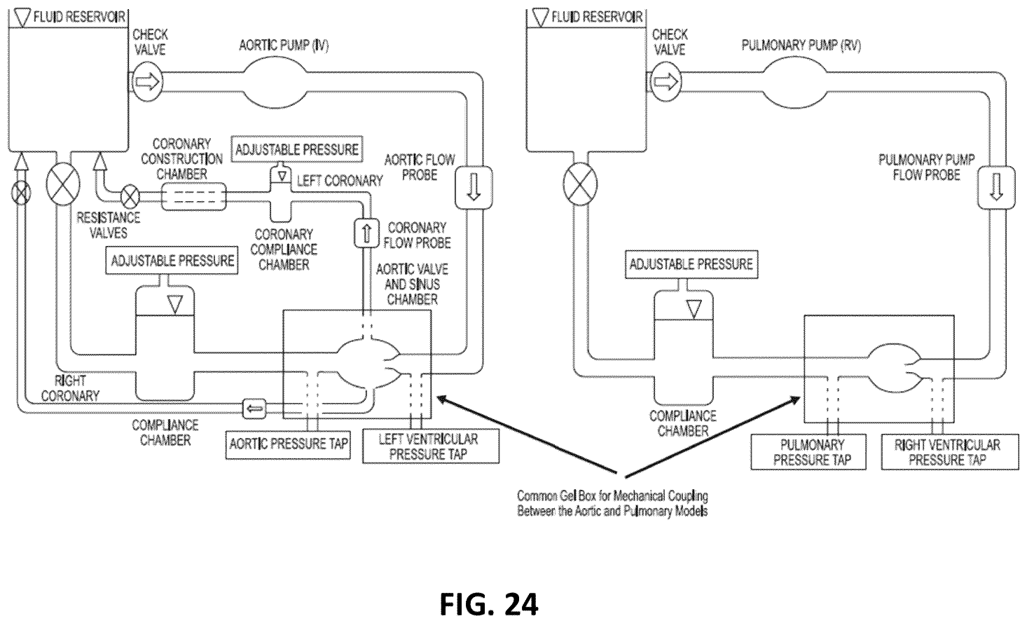

The 3D printed model is connected to a custom biventricular pulse duplicator system. FIG. 24 shows a schematic of the Bi-ventricular pulse duplicator system including two independent flow loops with mechanically coupled pulmonary and aortic models in the gel box. The bi-ventricular pump is coupled between two independent left and right heart simulators. Bioprosthetic valves provide the valvular functions necessary for the left and right circuit to operate physiologically. Viscosity matched water-glycerin blood analog is used as the flow loop fluid. The flow loop is initially tuned to individual patient's baseline hemodynamic condition as well as altered hemodynamic conditions. The baseline hemodynamic condition corresponds to a cardiac output of 3.5 L/min/m² based on patient's body surface area (BSA), and an average left coronary flow rate of 3.5% and right coronary flow rate of 1.5% of cardiac output with the resting blood pressure based on the patient's cuff pressure prior to CTA. Tuning the flow loop to achieve these hemodynamic parameters sets the corresponding resistances and compliances both in the coronary and main arterial circuits. Altered hemodynamic conditions correspond to two specific sets of experiments: (1) simulated exercise condition with 2× and 4× increase in cardiac output as well as left and right coronary flow by changing heart rate to 120 beats per minute (bpm) and 140 bpm respectively; and (2) starting with baseline conditions and maintaining cardiac output, increase the mean aortic blood pressure to a maximum of 200 mmHg while continuously recording coronary flow rate. In addition, in a subset of patients, FFR and flow testing are incorporated at different physiological states to study the effect of systolic and diastolic pressure, instantaneous flow rate, cardiac output, and left ventricular end-diastolic pressure (LVEDP) on FFR/pressure in the proximal anomalous coronary artery. The responses to upstream and downstream physiological variables provide control and understanding of the impact of hemodynamic conditions that may occur only during exercise.

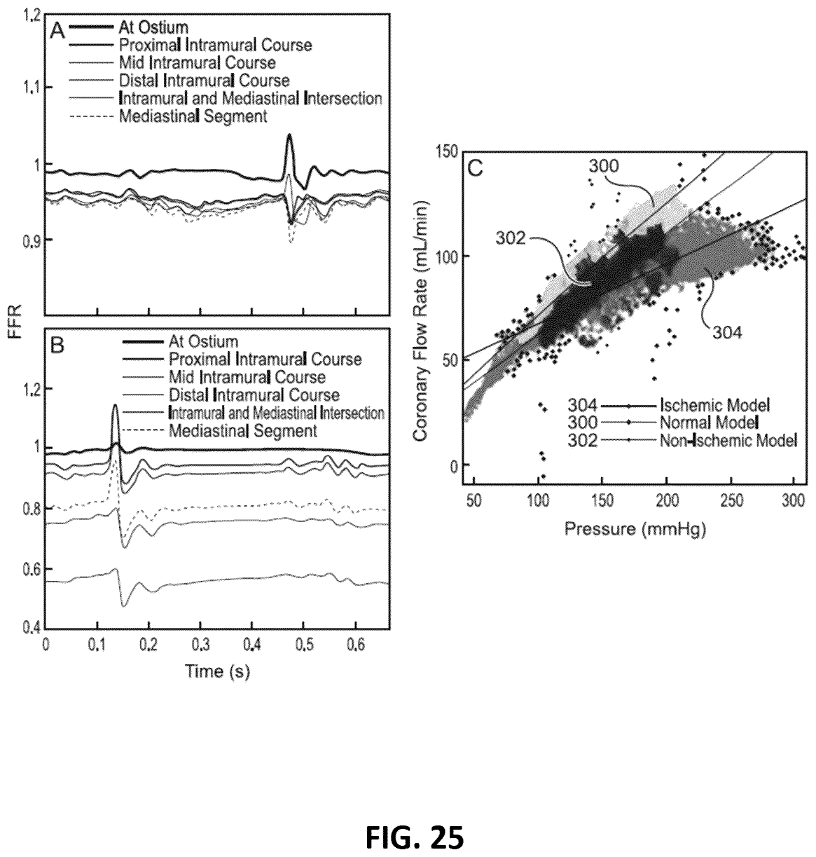

Using this setup, 3D-printed patient specific flow studies are performed in 10 models in 6 subjects using the validated approach, 3 of which are illustrated in FIG. 8, including (1) 9-year-old male with AAOCA but without ischemia; (2) 11-year-old male with AAOCA and confirmed ischemia on stress testing; and (3) 16-year-old male with normal coronary artery anatomy. FFR was computed as the ratio of distal pressure to aortic pressure. An FFR value lower than 0.8 is considered as the threshold for experimental ischemia. In FIG. 25, plots A, B, and C show, respectively, a non-ischemic model, an ischemic model, and an instantaneous coronary flow rates (IFR) as the aortic pressure is increased during simulated exercise. A simulated stress test is performed to increase the aortic pressure from about 100 mmHg to about 200 mmHg to observe effects of pressure-driven anatomical deformation on coronary flow. The normal coronary shows 100% flow without any change in FFR. For the patient with AAOCA with confirmed ischemia, FFR drops below the 0.8 threshold (see plot B), whereas, in the patient without inducible ischemia, the FFR does not drop below 0.9 (see plot A). The simulated stress test shows the variations of coronary flow rate with increasing aortic pressure indicating different distribution patterns for each patient (see plot C). A linear fit is used to compare the slopes of the curves. The normal model 300 shows the highest slope (0.5438) followed by the non-ischemic model 302 (slope=0.4706). The smallest slope of 0.2846 is found in the ischemic model 304.

These data provide support to the hypothesis that the risk of SCD in AAOCA is driven by biomechanically driven narrowing of the proximal coronary artery during exercise, causing sudden reduction of coronary flow and FFR. This has also been demonstrated in small clinical series using catheterization-based FFR. This sets the overarching central hypothesis for this proposal that the risk of SCD in AAOCA has a patient specific morphological basis and is driven by biomechanically driven narrowing of the proximal anomalous coronary during exercise.

There are morphologically based mechanisms that are associated with hemodynamic differences between AAOCA patients with ischemia when compared to those without. One or more of these mechanisms is dynamic and changes sufficiently in exercise to cause changes in experimental FFR.

The biomechanical model is configured to reveal key biomechanical mechanisms in a variety of patient-specific models and may be refined to better capture material properties of the aortocoronary tissue and the preload-afterload conditions. In addition, the morphological substrate may also be incorporated into the patient-specific 3D-printed model. Retrospectively electrocardiogram (ECG)-gated CTA data from each AAOCA subject may be segmented for the aortic valve, proximal aorta, and proximal right and left coronary arteries, and 3D printed models may be created with connections to inflow and outflow pipes. The same process may be repeated for the pulmonary artery.

The measurements (e.g., the peripheral intravenous line (PIV), IVUS, FFR, and instantaneous wave-free ratio (IFR) measurements) are hypothesis specific based on the mechanisms. For FFR characterization, dynamic pressure waveforms may be acquired over 100 cardiac cycles at 100 Hz using a Millar catheter (0.84 Fr Millar Mikro-Tip®) or a wire transducer (in small caliber coronaries). The pressure tracings may be ensemble averaged and the ratio of the pressure to the most upstream measurement location (aortic pressure) is computed. This ratio is the dynamic FFR which may be plotted as a function of location along the anomalous coronary starting with upstream of the ostium (where FFR=1.0) through the intra-mural course and then through the mediastinal course to identify location of FFR drop. This approach enables visualizing any pressure recovery and screening for dynamic changes (e.g. when luminal collapse occurs).

For coronary flow characterization, the instantaneous flow rate, quantitative coronary angiography (QAC) of the anomalous coronary is measured at 100 Hz using a transonic flow probe. This flow rate waveform is “set” during the tuning of the flow loop but is an independent measurement during the altered hemodynamic conditions where the model is subjected to simulated exercise as well as titrated high blood pressure conditions. Plots of coronary flow as a function of aortic pressure are generated as a “dose” curve to assess dynamic changes in resistance of the anomalous coronary. Resistance measures are calculated as the slope of the curve and compared between groups.

For dynamic caliber characterization using IVUS, as with FFR measurements at discrete points traversing the anomalous coronary artery, measurements are performed using the IVUS catheter. A continuous recording of luminal caliber starting with the ostium through the mediastinal course of the artery is performed. Dynamic luminal area plots at each FFR measurement location are plotted as a function of space as well as time.

For 3D particle tracking velocimetry (PTV), detailed measurements of the velocity field and vorticity field may be acquired in the region over the proximal coronary artery. These models are built from silicone molding process as opposed to Agilus for optical clarity, while preserving pulsatile properties. The methods of the present embodiments include the use of the 3D tomographic peripheral intravenous line (PIV) system (available from LaVision, Germany) for data acquisition and processing. The flow loop fluid is seeded with 1-10 microns melamine resin particles coated with Rhodamine-B. The Nd: YLF Dual Cavity Diode Pumped Solid State High Repetition Rate Laser (available from Photonics Industries, Bohemia, NY, U.S.A.) is used for volume illumination through the lumen of the intra-mural section of the anomalous artery. A four-frame high-speed CMOS camera (available from Photronix Inc.) is used to obtain volumetric tomographic measurements spanning the artery and record 1000 phases over the cardiac cycle. Detailed characterization of the data which includes viscous as well as turbulent stresses is performed with the protocols and algorithms.

For 4D flow MRI, in a subset of patients, 4D flow MRI of the 3DP models are performed, and the results are compared to PIV using a custom 3 Tesla magnetic resonance imaging (3T MR) compatible twin system of the flow-loop.

### Statistical Evaluation of Mechanistic Basis of Ischemia in AAOCA

For power analysis, statistical power calculation is performed based on the primary outcome of the tests set up for validating the biomechanical basis of ischemia in AAOCA. A total sample size of 110 with 50 samples from the ischemic subgroup, 50 samples from non-ischemic group and 10 normal controls, achieves 80% power to detect an odds ratio of 1.52 and 2.00 with respect to the intra-cluster correlation of 0.7 (moderate correlation) and 0.2 (weak correlation). The proportion of experimental ischemia in non-ischemia group is assumed to be 0.5 under the alternative hypothesis. The test statistic used is the effect regression coefficient from a mixed-effects logistic regression model (significance 0.050).

Although example aspects of the present disclosure are explained in some instances in detail herein, it is to be understood that other aspects are contemplated. Accordingly, it is not intended that the present disclosure be limited in its scope to the details of construction and arrangement of components set forth in the following description or illustrated in the drawings. The present disclosure is capable of other aspects and of being practiced or carried out in various ways.

As used herein, the term “or” may be construed in either an inclusive or exclusive sense. Moreover, the description of resources, operations, or structures in the singular shall not be read to exclude the plural. Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps.

Terms and phrases used in this document, and variations thereof, unless otherwise expressly stated, should be construed as open ended as opposed to limiting. Adjectives such as “conventional,” “traditional,” “normal,” “standard,” “known,” and terms of similar meaning should not be construed as limiting the item described to a given time period or to an item available as of a given time, but instead should be read to encompass conventional, traditional, normal, or standard technologies that may be available or known now or at any time in the future. The presence of broadening words and phrases such as “one or more,” “at least,” “but not limited to” or other like phrases in some instances shall not be read to mean that the narrower case is intended or required in instances where such broadening phrases may be absent.

The foregoing description of the present disclosure has been provided for the purposes of illustration and description. It is not intended to be exhaustive or to limit the disclosure to the precise forms disclosed. The breadth and scope of the present disclosure should not be limited by any of the above-described exemplary embodiments. Many modifications and variations will be apparent to the practitioner skilled in the art. The modifications and variations include any relevant combination of the disclosed features. The embodiments were chosen and described in order to best explain the principles of the disclosure and its practical application, thereby enabling others skilled in the art to understand the disclosure for various embodiments and with various modifications that are suited to the particular use contemplated. It is intended that the scope of the disclosure be defined by the following claims and their equivalence.

In one aspect, a method may include an operation, an instruction, and/or a function and vice versa. In one aspect, a clause or a claim may be amended to include some or all of the words (e.g., instructions, operations, functions, or components) recited in other one or more clauses, one or more words, one or more sentences, one or more phrases, one or more paragraphs, and/or one or more claims.

To illustrate the interchangeability of hardware and software, items such as the various illustrative blocks, modules, components, methods, operations, instructions, and algorithms have been described generally in terms of their functionality. Whether such functionality is implemented as hardware, software or a combination of hardware and software depends upon the particular application and design constraints imposed on the overall system. Skilled artisans may implement the described functionality in varying ways for each particular application.

The functions, acts or tasks illustrated in the Figures or described may be executed in a digital and/or analog domain and in response to one or more sets of logic or instructions stored in or on non-transitory computer readable medium or media or memory. The functions, acts or tasks are independent of the particular type of instructions set, storage media, processor or processing strategy and may be performed by software, hardware, integrated circuits, firmware, microcode and the like, operating alone or in combination. The memory may comprise a single device or multiple devices that may be disposed on one or more dedicated memory devices or disposed on a processor or other similar device. When functions, steps, etc. are said to be “responsive to” or occur “in response to” another function or step, etc., the functions or steps necessarily occur as a result of another function or step, etc. It is not sufficient that a function or act merely follow or occur subsequent to another. The term “substantially” or “about” encompasses a range that is largely (anywhere a range within or a discrete number within a range of ninety-five percent and one-hundred and five percent), but not necessarily wholly, that which is specified. It encompasses all but an insignificant amount.

As used herein, the phrase “at least one of” preceding a series of items, with the terms “and” or “or” to separate any of the items, modifies the list as a whole, rather than each member of the list (e.g., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The word “exemplary” is used herein to mean “serving as an example, instance, or illustration.” Any embodiment described herein as “exemplary” is not necessarily to be construed as preferred or advantageous over other embodiments. Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A reference to an element in the singular is not intended to mean “one and only one” unless specifically stated, but rather “one or more.” The term “some” refers to one or more. Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description. No claim clement is to be construed under the provisions of 35 U.S.C. § 112 (f) unless the clement is expressly recited using the phrase “means for” or, in the case of a method claim, the element is recited using the phrase “step for.”

While this specification contains many specifics, these should not be construed as limitations on the scope of what may be claimed, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the claimed subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirements of the applicable patent law, nor should they be interpreted in such a way.

What is claimed is:

1. A computer-implemented method, comprising:
receiving medical imaging data of a patient;
extracting, from the medical imaging data, patient-specific morphological imaging biomarkers pertaining to anomalous aortic origin of a coronary artery (AAOCA);
incorporating the biomarkers into a computer model;
simulating, using the computer model, hemodynamics of the patient under simulated stress conditions and a combination of variables, the variables comprising physiological properties of the patient; and
predicting and outputting a patient-specific risk profile of ischemia and/or sudden cardiac death based on simulated hemodynamics.

2. The computer-implemented method of claim 1, wherein the computer model comprises a reduced order model that incorporates the biomarkers and solves for the hemodynamics comprising dynamic pressure and/or flow changes from ostium through distal coronary artery under the simulated stress conditions.

3. The computer-implemented method of claim 1, wherein the physiological properties comprise material properties of the patient's heart over an age span.

4. The computer-implemented method of claim 1, wherein the physiological properties comprise the biomarkers.

5. The computer-implemented method of claim 1, wherein the simulated stress conditions comprise flow and/or stress conditions of the patient at rest and during exercise.

6. The computer-implemented method of claim 1, further comprising performing sensitivity analysis to determine sensitivity scores of the variables on the hemodynamics.

7. The computer-implemented method of claim 1, further comprising predicting a dominating mechanism of ischemia and/or sudden cardiac death in AAOCA based on the simulated hemodynamics.

8. The computer-implemented method of claim 1, further comprising outputting simulated changes in FFR, iFR, and/or percent flow for a range of physiological properties.

9. The computer-implemented method of claim 1, wherein the medical imaging data comprise computed tomography (CT), magnetic resonance imaging (MRI), and/or ultrasound aortic and coronary imaging data of the patient.

10. The computer-implemented method of claim 1, wherein the biomarkers comprise coronary ostial location, ostial branching pattern, ostial shape, proximal coronary caliber, intramural length, interarterial length, thickness of the intercoronary pillar, thickness of the intimal wall, or a combination thereof.

11. The computer-implemented method of claim 1, further comprising performing principal component analysis (PCA) on the medical imaging data to extract the biomarkers.

12. The computer-implemented method of claim 1, further comprising deriving Z-scores of variation in the biomarkers in AAOCA.

13. The computer-implemented method of claim 1, further comprising using K-means clustering to characterize discriminatory power of imaging indices and proximal coronary shape for ischemia through a matching matrix.

14. A non-transitory machine-readable storage medium comprising machine-readable instructions for causing a processor to execute a method for predicting risk of ischemia in AAOCA, the method comprising:
receiving medical imaging data of a patient;
extracting, from the medical imaging data, patient-specific morphological imaging biomarkers pertaining to anomalous aortic origin of a coronary artery (AAOCA);
incorporating the biomarkers into a computer model;
simulating, using the computer model, hemodynamics of the patient under simulated stress conditions and a combination of variables, the variables comprising physiological properties of the patient; and
predicting and outputting a patient-specific risk profile of ischemia and/or sudden cardiac death based on simulated hemodynamics.

15. The non-transitory machine-readable storage medium of claim 14, wherein the computer model comprises a reduced order model that incorporates the biomarkers and solves for the hemodynamics comprising dynamic pressure and/or flow changes from ostium through distal coronary artery under the simulated stress conditions, and the physiological properties comprise material properties of the patient's heart over an age span and the biomarkers.

16. The non-transitory machine-readable storage medium of claim 14, wherein the method further comprises performing sensitivity analysis to determine sensitivity scores of the variables on the hemodynamics.

17. The non-transitory machine-readable storage medium of claim 14, wherein the method further comprises predicting a dominating mechanism of ischemia and/or sudden cardiac death in AAOCA based on the simulated hemodynamics.

18. The non-transitory machine-readable storage medium of claim 14, wherein the method further comprises comprising outputting simulated changes in FFR, iFR, and/or percent flow for a range of physiological properties.

19. The non-transitory machine-readable storage medium of claim 14, wherein the medical imaging data comprise computed tomography (CT), magnetic resonance imaging (MRI), and/or ultrasound aortic and coronary imaging data of the patient, and wherein the biomarkers comprise coronary ostial location, ostial branching pattern, ostial shape, proximal coronary caliber, intramural length, interarterial length, thickness of the intercoronary pillar, thickness of the intimal wall, or a combination thereof.

20. The non-transitory machine-readable storage medium of claim 14, wherein the method further comprises performing principal component analysis (PCA) on the medical imaging data to extract the biomarkers.
